(19) United States
(12) Patent Application Publication
(10) Pub. No.: US 2026/0201296 A1
(43) Pub. Date: Jul. 16, 2026

(54) SURFACES FOR CONTROLLED CELL ADHESION AND RELATED METHODS

(71) Applicants: Massachusetts Institute of Technology, Cambridge, MA (US); The Broad Institute, Inc., Cambridge, MA (US)

(72) Inventors: Kripa K. Varanasi, Lexington, MA (US); Caroline Taylor McCue, Cambridge, MA (US); Yuen-Yi Tseng, Melrose, MA (US); Adel Atari, Cambridge, MA (US)

(73) Assignees: Massachusetts Institute of Technology, Cambridge, MA (US); The Broad Institute, Inc., Cambridge, MA (US)

(21) Appl. No.: 19/133,506

(22) PCT Filed: Nov. 28, 2023
(86) PCT No.: PCT/US2023/081432

§ 371 Date: May 28, 2025

Related U.S. Application Data
(60) Provisional application No. 63/385,235, filed on Nov. 29, 2022.

(51) Int. Cl. C12M 1/00; C12M 1/42

(52) U.S. Cl. CPC C12M 23/20; C12M 23/22; C12M 35/02

(57) ABSTRACT

According to some embodiments, the cell culture system allows for the passage of cells in high-throughput cell culture applications. Unlike enzymatic detachment processes, the cell culture system, in some embodiments, limits any imparted damage onto the cell. Additionally, the lack of reliance on enzymes for cell detachments (e.g. trypsin) reduces and/or prevents the accrual of genetic mutations that can occur during enzymatic detachment processes. The cell culture system, in some embodiments, is capable of passaging and/or detaching cells at rates convenient for high throughput cell culturing and/or automated cell culturing regimes. Advantageously, the cell culture system, in certain embodiments, provides sufficient adhesion to promote cell growth and proliferation, but is capable of reducing the adhesion between the cell and the surface upon application of the electric field.

### RELATED APPLICATIONS

This application claims priority under 35 U.S.C. § 119(e) to U.S. Provisional Patent Application No. 63/385,235, filed Nov. 29, 2022, and entitled “SURFACES FOR CONTROLLED CELL ADHESION,” which is incorporated herein by reference in its entirety for all purposes.

### TECHNICAL FIELD

The use of surfaces to control cell adhesion is generally described.

### BACKGROUND

Culturing cells is a laboratory task involved in essentially all research that involves cells. However, trypsinization, the primary method used to passage cells, can be tedious for researchers, creates liquid and solid waste, and can cause damage to cells. The ability to culture cells without the need to use trypsin (or similar chemicals, or other methodologies) to detach adherent cells from cell culture dishes can save time for researchers, streamline the process of cell culture, and improve the outcomes of cell line development. A fast platform for on-demand cell detachment would benefit researchers that work with primary cells and would have impacts in cancer treatment, specifically in developing and manufacturing cell-based therapeutics and personalized medicine by enabling primary cells from tumors to be more easily cultured in labs, possible treatments to tested in vitro, and by reducing the complexity of the process for culturing cells used in personalized treatments.

Cell-based assays are a common biological experiment for biological research labs across a wide range of fields. Many researchers perform cell culture tasks daily at large scale. Additionally, for many groups that work with primary cells, damage to cells during subculture caused by trypsin, or other species or methodologies, can result in lower success rates of cell line development and can affect the integrity of the cells being grown. Although some alternative gentle dissociation enzymes exist, such as Accutase and TrypLE, they have a long neutralization process (Accutase takes around 45 minutes), which further increases cell culture process time. Additionally, some cell types do not dissociate well with these gentler dissociation enzymes. Existing non-enzymatic approaches include thermal-responsive polymer coatings (such as Isurtherm), which dissolve at lower temperatures, enabling cells to detach as sheets or clumps, which is gentler on cells than dissociation enzymes, however, the process is far slower as well, taking about 2 hours for cells to detach, and requires cells to be kept at room temperature for long periods of time, which may be damaging to certain cell types. Other thermo-responsive techniques have been shown to work faster but result in much lower cell growth and proliferation over time.

Therefore, an improved and effective technology, including processes, articles, systems, etc., for cell detachment for various cell types, including cancer cell lines, patient-derived cultures, primary cells, and immortalized different cell lineage normal cells, whether used alone or in combination with existing methodologies such as those noted above, would greatly benefit researchers who use cell culture.

### SUMMARY

The subject matter of the present disclosure involves, in some cases, interrelated products, alternative solutions to a particular problem, and/or a plurality of different uses of one or more systems and/or articles.

In one aspect, cell culture systems are described. In some embodiments, a cell culture system configured to selectively affect cell adhesion and/or detachment, comprising a surface suitable for cell culture, the surface comprising a redox active species; an electric system configured to apply an alternating electrical field at or proximate the surface at an alternating frequency greater than or equal to greater than or equal to 0.01 Hz and less than or equal to 0.1 Hz.

In certain embodiments, a cell culture system, comprising a vessel capable of being at least partially filled with a cell culture medium, the vessel comprising a first electrode in electrical communication with the cell culture medium when present; and a surface, within the vessel and at least partially submerged with the cell culture medium when present, the surface being electrically coupled to the first electrode and configured to reduce the adhesion between the surface and cultured cells on the surface upon application of an AC voltage at a frequency capable of altering the morphology of cultured cells, wherein the surface comprises a redox active species.

In another aspect, methods for detaching cells from a surface are provided. In some embodiments, a method for detaching cells from a surface comprising applying an AC voltage at a frequency capable of altering the morphology of cultured cells across a first electrode and a surface, such that the adhesion between the surface and cultured cells on the surface is reduced, wherein the first electrode is in electrical communication with the cell culture medium; the surface comprises a redox active species; and the surface is at least partially submerged with a cell culture medium when present.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1. A schematic depicting a cell culture system, according to some embodiments.

FIG. 2. A schematic depicting a cell culture system prior to the application of an AC voltage and/or alternating electric field, according to some embodiments.

FIG. 3. A schematic depicting a cell culture system during the application of an AC voltage and/or alternating electric field, according to some embodiments.

FIG. 4. A schematic depicting a three-dimensionally patterned surface for cell culturing, according to some embodiments.

FIG. 5. A schematic depicting a cell culture system comprising a three-dimensionally patterned surface and electrically conductive polymer network prior to the application of an AC voltage and/or alternating electric field, according to some embodiments.

FIG. 6. A schematic depicting a cell culture system comprising a three-dimensionally patterned surface and electrically conductive polymer network after the application of an AC voltage and/or alternating electric field, according to some embodiments.

FIG. 7A. Schematic showing the concept of how microtexture can reduce cell-surface adhesion by reducing cell-surface contact area, according to some embodiments.

FIG. 7B. Schematic showing our microtextured fabrication process, according to some embodiments.

FIG. 7C. Image of a 6 well cell culture plate with micromolded surfaces fabricated using our process, optical image of 5 μm micropost surfaces at 10× magnification (scale bar is 50 μm) and SEM image of the 5 μm micropost surface (scale bar is 10 μm), according to some embodiments.

FIG. 7D. SEM images of MG-63 cells grown on a flat polystyrene surface and the 2.5 μm post surface at 1000× magnification and 30° tilt (scale bars are 10 μm), according to some embodiments.

FIG. 8A. Schematic of the microfluidic set-up used to apply shear to cells, according to some embodiments.

FIG. 8B. Images of cells grown within the microfluidic chip before shear force is applied and after shear has been applied for 90 seconds with overlay showing cell identification (magnification=5×), according to some embodiments.

FIG. 8C. Time lapse graphs of the area of adhered cells on each surface over 90 seconds as 1.5 nN of shear force is applied, according to some embodiments.

FIG. 8D. Percent of cells remaining adhered after 2 minutes of 1.5 nN shear force (n=3), according to some embodiments.

FIG. 9A. Optical images at high and low cell density, according to some embodiments.

FIG. 9B. Plot of cell viability, according to some embodiments.

FIG. 9C. Plot of doubling time, according to some embodiments.

FIG. 9D. Ki67 staining (in green), according to some embodiments.

FIG. 9E. Plot of cell proliferation (Ki67 expression), according to some embodiments.

FIG. 9F. Plot of cell death assay results for MG63 and LNCaP cells grown on micromolded polystyrene surfaces, according to some embodiments.

FIG. 10. A) Schematic of how our voltage application system works to detach cells.

B) Image of a PEDOT:PSS-infused microtextured polystyrene surface.

C) Prototype of the voltage application system cell culture well.

D) Applying voltage to the PEDOT:PSS-infused surface with cell culture media inside the well.

FIG. 11A. Image of cells adhered to a PEDOT:PSS-infused microtextured polystyrene surface, according to some embodiments.

FIG. 11B. Cells detaching after 7.5V applied to the surface for 5 minutes (5× magnification), according to some embodiments.

FIG. 12A. Schematic of cell detachment on PEDOT:PSS surfaces, according to some embodiments.

FIG. 12B. Cyclic Voltammetry curve for PEDOT:PSS surface, with photograph indicating surface in reduced state (top), neutral state (middle) and in a oxidized state (bottom), according to some embodiments.

FIG. 12C. Image of voltage application to PEDOT:PSS surface, according to some embodiments.

FIG. 12D. Brightfield images showing impact of voltage application (5 VAC) on cells adhered to PEDOT:PSS surface (20× magnification), according to some embodiments.

FIG. 13A. Cells before and after 5 minutes of 5V DC or AC applied, and the corresponding graphs of the voltage and current applied to cells over time, according to some embodiments.

FIG. 13B. Current vs Voltage curves for PEDOT:PSS surfaces with MG63 cells adhered with sinusoidal voltage applied at frequencies of 0.1 Hz, 0.05 Hz, 0.025 Hz and 0.01 Hz, according to some embodiments.

FIG. 13C. Time lapse of brightfield images showing cell shape change with application of 5 VAC (f=0.05 Hz), 20× magnification, according to some embodiments.

FIG. 13D. Time lapse of brightfield images showing cell shape change in 10 mM HCl, 5× magnification, according to some embodiments.

FIG. 14A. Brightfield images (5× magnification) of MG63 human osteosarcoma cells after 48 hours on a polystyrene surface, according to some embodiments.

FIG. 14B. Brightfield images (5× magnification) of MG63 human osteosarcoma cells after 48 hours on a PEDOT:PSS surface, according to some embodiments.

FIG. 14C. Brightfield images (5× magnification) of MG63 human osteosarcoma cells after 5 minutes of 5V AC application on a PEDOT:PSS surface, according to some embodiments.

FIG. 14D. Brightfield images (5× magnification) of MG63 human osteosarcoma cells 48 hours after exposure to voltage, according to some embodiments.

FIG. 14E. Cell viability results, according to some embodiments.

FIG. 14F. Doubling time results for cells grown on polystyrene surfaces, PEDOT:PSS surfaces and exposed to 5V AC on PEDOT:PSS surfaces, according to some embodiments.

FIG. 15A. Schematic showing how cells were loaded into microfluidic chips, and then how voltage and shear were applied to cells.

FIG. 15B. Brightfield images of cells grown within the microfluidic chip before shear force is applied and after shear has been applied for 90 seconds (magnification=5×), according to some embodiments.

FIG. 15C. Time lapse graphs of the area of adhered cells on each surface over 90 seconds as 1.5 nN of shear force is applied, according to some embodiments.

FIG. 16. Cyclic voltammetry curves for PEDOT:PSS within different electrolytes, according to some embodiments.

### DETAILED DESCRIPTION

Systems, articles, and methods for controlling cell adhesion are generally described, in which a cell culture arrangement can be configured to selectively affect cell adhesion and/or detachment. In one set of embodiments, cell adhesion and/or detachment can be affected by an applied electric field and/or a change in ionic environment proximate a cell(s), optionally via application of an electric field. In some embodiments, upon the application of an alternating electric field and/or AC voltage at or proximate the surface, a redox reaction associated with the surface, a cell culture medium, and a first electrode at least partially submerged in the cell culture medium may induce a change in morphology of cells adhered on the surface. This change in morphology can reduce the adhesion between the cell and the surface, and in some cases, lead to cell detachment from the surface.

Conventional methods used to passage and/or detach cells from surfaces often involve the use of enzymatic processes such as trypsinization. However, while the use of enzymes is widely used throughout cell culture processes, it can often be labor-intensive, produce large quantities of liquid waste, and result in the accumulation of undesirable and/or unintentional genetic mutations of the cultured cells. The inefficiency of enzymatic detachment processes is especially a problem in high-throughput cell culture settings. Additionally, enzymatic process can damage cell surface receptors and other protein-level features thereby limiting proteomic analysis fidelity and accuracy. High-throughput cell culture systems may require more effective and efficient cell detachment systems that do not rely on enzymatic activity.

Surprisingly, as described in this disclosure, the inventors have discovered a cell culture system that can reduce adhesion and/or promote detachment of cells adhered to surfaces (e.g. a surface of a cell culture vessel). The cell culture system, according to some embodiments, comprises a surface suitable for cell culture and an electric system configured to apply an electric field at or proximate the surface. Upon application of the electric field, cells adhered to the surface may undergo a change in morphology thereby reducing the adhesion strength between the cell and the surface and facilitating the detachment and/or passage of the cells.

According to some embodiments, the cell culture system allows for the passage of cells in high-throughput cell culture applications. Unlike enzymatic detachment processes, the cell culture system, in some embodiments, limits any imparted damage onto the cell. Additionally, the lack of reliance on enzymes for cell detachments (e.g. trypsin) reduces and/or prevents the accrual of genetic mutations that can occur during enzymatic detachment processes. The cell culture system, in some embodiments, is capable of passaging and/or detaching cells at rates convenient for high throughput cell culturing and/or automated cell culturing regimes.

Advantageously, the cell culture system, in certain embodiments, provides sufficient adhesion to promote cell growth and proliferation, but is capable of reducing the adhesion between the cell and the surface upon application of an alternating electric field and/or an AC voltage so that the cultured cells can be passaged appropriately. That is, the adhesion between the surface and culture cells can be controlled and/or modulated to efficiently culture and passage cells.

In order to develop a system that can simplify and speed up the process of cell passaging, a platform that triggers cell detachment in response to simple external stimuli, such as a voltage change or a change in mechanical properties, was designed and tested. There are many commercial applications of these cell culture platforms in high throughput cell culture, tissue engineering, cell therapy manufacturing and personalized medicine. This platform is more amenable to automation than the current process for cell passaging. Due to the many issues with trypsin and similar chemicals damaging cells, using this technique would be beneficial for culturing primary cells in clinical applications such as growing skin grafts, growing iPSCs for individual patients and for manufacturing the cells needed for cell-based treatments such as CAR-T therapy. It would also be applicable to medical research using cells from patient tumor biopsies, such as for testing potential treatments in vitro without introducing any other chemicals that could impact the results of such experiments. In addition, this technique could be useful for altering electrical properties of cells and could potentially facilitate electroporation.

A major advantage of this platform over other mechanical and non-trypsin approaches to cell passaging is that it would require little modification of existing cell culture processes. The piece of equipment required beyond existing cell culture equipment would be, but is not limited to, a power supply, which is readily available in most laboratories. In addition, fewer consumables and less active human labor would be required, making this a more sustainable alternative to trypsin-based cell passaging.

Without wishing to be bound by any particular theory, cells adhered to the surface may undergo a morphology change from a flat structure while growing and/or proliferating to a more rounded structure upon application of the electric field in response to a flux of H⁺ at the cell-surface interface. Ion channels within the cell, in some embodiments, may be sensitive to the flux of H⁺ generated by the redox reaction and change into a more rounded morphology reducing the contact area between the cell and the surface. The reduction in adhesion between cultured cells and the surface and/or the detachment of cultured cells from the surface may be highly dependent on the change in ion concentration at the interface between the cultured cell and the surface. That is, a greater change in the concentration of ions at the aforementioned interface will lead to a greater reduction in adhesion. The frequency of the alternating electric field and/or AC voltage may be at least partially responsible for inducing the flux of ions between the cell culture medium and the surface. Relatively high frequencies may not provide sufficient time for ions to migrate between the cell culture medium and the surface and may not result in controllable cell adhesion. However, advantageously, lower frequencies can, in some embodiments, allow for sufficient ion flux to change the morphology of the cell and sufficiently reducing adhesion to allow for the passage of cells.

According to some embodiments, the cell culture system selectively affects cell adhesion and/or detachment upon the application of voltage by inducing a morphology change on the surface. The morphology change on the surface can, in some embodiments, dislodge cells from the surface in response to the application of an alternating electric field and/or a AC voltage. Without wishing to be bound by any particular theory, the application of an electric field at or proximate the surface, in some instances, can induce swelling of some portions of the surface. The portions of the surface that undergo swelling, in some embodiments, be directionally controlled via a three-dimensionally patterned surface. Without the application of an electric field, the three-dimensionally patterned surface can reduce the adhesion between cells and the surface by reducing the effective contact area between the cell and the surface as exhibited in Equation 1 and Example 2. Upon application of the electric field and/or the voltage, cells adhered to the surface can be dislodged from the surface due to reduced adhesion between the cell and the three-dimensional patterned surface and the morphological change experienced by swollen portions of the surface. Accordingly, the cell culture system allows for cell adhesion can be controlled to promote cell growth, proliferation, and detachment.

In some embodiments, the cell culture system comprises a surface. The surface can, in some embodiments, be suitable for cell culture. That is, the surface may have properties (e.g. biocompatibility, roughness, or adhesion) that may allow for the growth and proliferation of any of a myriad of cell strains. A person of ordinary skill in the art may be capable of determining whether a surface is suitable for cell growth by conducting screening tests on a particular surface to determine the viability of cell growth. A surface that is suitable for cell growth may limit and/or prevent cell damage during growth and proliferation. Characteristics of a surface that may be suitable for cell growth include, but are not limited to, limited external stress applied to the cell by the surface (e.g. excessive surface roughness), biocompatibility, sufficient adhesion for cell growth and/or proliferation, and limited adhesion for eventual detachment of cells from the surface. In some embodiments, the cell surface is biocompatible. That is, the surface in the context of this disclosure does not negatively affect the viability of cell growth and proliferation. In some embodiments, the cell surface is relatively flat and does not impart undue stress onto growing cell. Undue stress imparted onto growing cells may lead to limited growth and proliferation. In some embodiments, the cell surface has sufficient adhesion to allow for cell growth and proliferation, but does not provide excessive adhesion that may harm and/or damage the cell during removal. Proliferation of cells on a particular surface can be determined using procedures described in Examples 1 and 2, but can be generally by the doubling time (e.g. the duration of time that is require for a cell culture to double in number of cells).

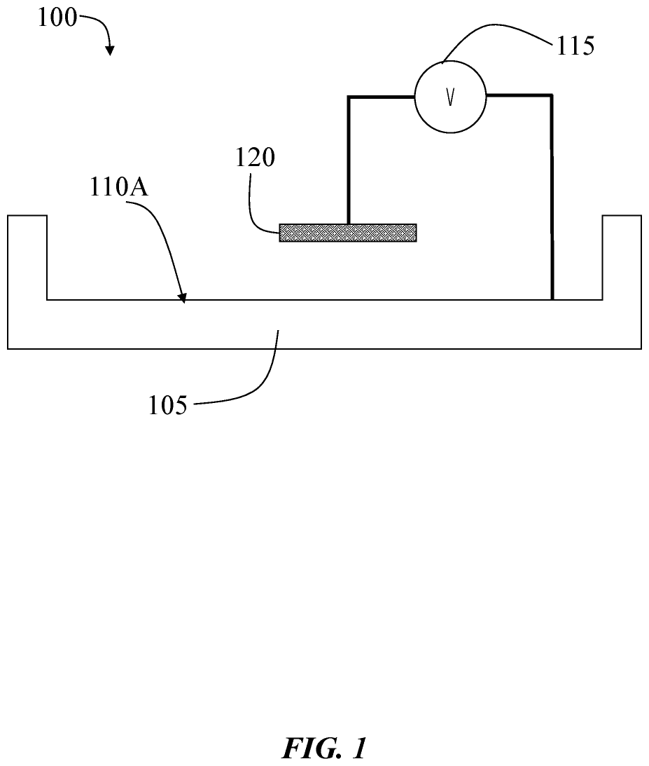

According to some embodiments, the surface suitable for cell culture is a surface within a vessel. For example, in FIG. 1, vessel 105 comprises surface 110A that is suitable for cell culture. In some embodiments, the vessel can be any of myriad of cell culture vessels (e.g. petri dishes, bioreactors, flasks such as flat-sided tissue culture flasks, Erlenmeyer flasks, and spinner flasks). The vessel, in some embodiments, may be configured to culture cells. That is, the vessel may comprise characteristics suitable for cell growth (e.g. biocompatibility, adhesions, surface roughness, capable of being at least partially filled with cell culture medium), as described later in this disclosure.

In some embodiments, a surface suitable for cell culture has relatively high cell viability. Cell viability, another characteristics of a surface that may be suitable for cell growth, is a measure of the proportion of live, healthy cells within a particular population, and high cell viability of a surface may render that surface to be suitable for cell culture. In some embodiments, the cell culture system comprises a surface suitable for cell culture with cell viability greater than or equal to 80%, greater than or equal to 85%, greater than or equal to 90%, greater than or equal to 95%, or greater than or equal to 99%. In some embodiments, the cell culture system comprises a surface suitable for cell culture with cell viability less than or equal to 99%, less than or equal to 95%, less than or equal to 90%, less than or equal to 85%, or less than or equal to 80%. Combinations of these ranges are possible (e.g. greater than or equal to 80% and less than or equal to 80%). Other ranges are also possible. Cell viability can be measured by first seeding cell culture plates on surfaces of interest, trypsinizing the cells, and collecting the passaged cells for counting using an automated cell counter (e.g. CellDrop Automated Cell Counter). Cell viability is one of a myriad of tests to determine the suitability of a surface for cell culturing. Other tests (e.g. doubling time) may also be used to assess the suitability of a surface for cell culturing.

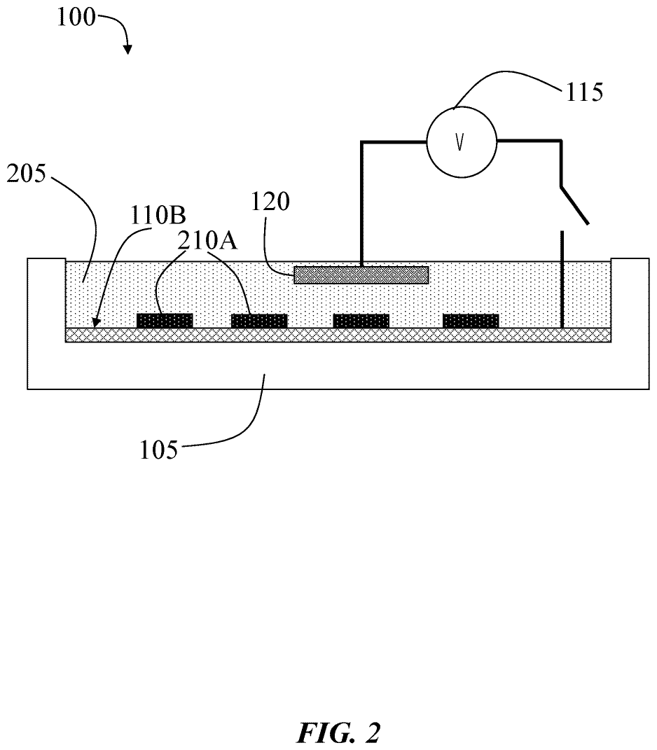

In some embodiments, the surface is at least partially submerged in a cell culture medium, when present. For example, in FIG. 2, cell culture system 100 comprises vessel 105 comprising surface 110B that is submerged in cell culture medium 205. In some embodiments, the surface is in contact with the cell culture medium so that the growing cells are provided sufficient nutrients for growth. In some embodiments, the surface is mostly submerged in the cell culture medium. In some embodiments, the surface is submerged in the cell culture medium. In some embodiments, the cell culture medium is not present in the cell culture system.

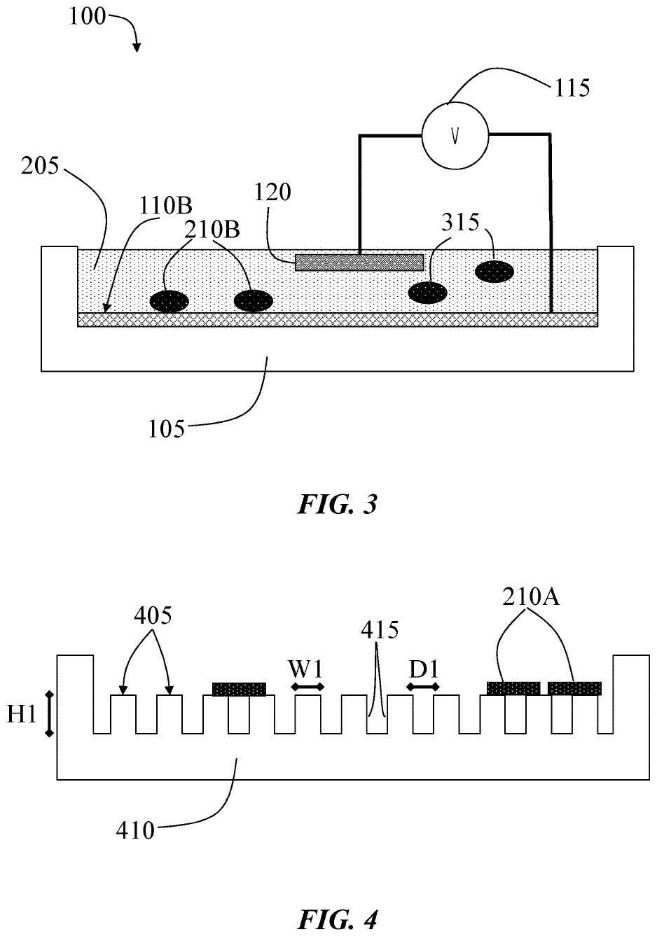

In order to control adhesion of cells on the surface via redox reaction, the surface, in some embodiments, is in electrical communication with a first electrode. For example, in FIG. 3, surface 110B is in electrical communication with first electrode 120 via electrical system 115. The alternating electrical field may be applied to at or proximate to the surface to induce a redox reaction that affects cell adhesion. Therefore, the surface may be in electrical communication with the first electrode to ensure the applied alternating field is at or proximate the surface. When a voltage is applied between the first electrode and the surface, an electrical field is created which can, in some embodiments, change the morphology of cultured cells on the surface thereby reducing the adhesion between the surface and the cultured cells. In some embodiments, the surface is electrically coupled to the first electrode. In some embodiments, the first electrode comprises platinum. In some embodiments, the first electrode comprises a mesh form factor. In some embodiments, the first electrode can be any of myriad of electrode materials suitable for redox reactions (e.g. platinum, titanium, gold, iridium, iron, silver, zinc, etc.).

In some embodiments, the first electrode is at least partially submerged in the cell culture medium. For example, in FIG. 2, first electrode 120 is submerged in cell culture medium 205. In some embodiments, the first electrode is in contact with the cell culture medium. In some embodiments, the first electrode is submerged in the cell culture medium.

In some embodiments, the surface is capable of participating in a redox reaction. That is, the surface, in some embodiments, comprises characteristics that allow render the surface electrochemically active (e.g. electrically conductive, redox active species). In some embodiments, the surface comprises an electrically conductive polymer. In some embodiments, the surface comprises poly(3,4-ethylenedioxythiophene) polystyrene sulfonate (PEDOT:PSS). In some embodiments, the surface comprises polypyrrole, polyaniline, polythiophene, and/or PEDOT:PSS. In some embodiments, the surface comprises any of a myriad of conducting polymers with conductivity sufficient to participate in the redox reaction between the first electrode, cell culture medium, and the surface. In some embodiments, the surface comprises a biocompatible, electrically conductive polymer.

In some embodiments, the electrically conductive polymer is cross-linked. In some embodiments, the surface comprises a cross-linker. In some embodiments, the surface comprises the electrically conductive polymer and a crosslinker that can be cross-linked together (e.g. cured) to form an electrically conductive polymer network. In some embodiments, the cross linker comprises poly(ethylene glycol)diglycidyl ether (PEGDE). In some embodiments, the crosslinker comprises any of a myriad of crosslinkers capable of crosslinking the electrically conductive polymer at relatively low temperatures and produce films of relatively high hydrophilicity and relatively high conductivity. A person having ordinary skill in the art, in view of the entirety of this disclosure, may be capable of determining suitable cross-linkers based on the temperature required to cure the electrically conductive polymer network, the resulting conductivity, the resulting hydrophilicity, and/or the biocompatibility of the network.

In some embodiments, the electrically conductive polymer network has a relatively high conductivity. In some embodiments, the electrically conductive polymer network has a conductivity greater than or equal to 500 S cm⁻¹. In some embodiments, the electrically conductive polymer network has a conductivity greater than or equal to 50 S cm⁻¹, greater than or equal to 100 S cm⁻¹, greater than or equal to 200 S cm⁻¹, greater than or equal to 300 S cm⁻¹, greater than or equal to 400 S cm⁻¹, greater than or equal to 500 S cm⁻¹, or greater than or equal to 1000 S cm⁻¹. In some embodiments, the electrically conductive polymer network has a conductivity less than or equal to less than or equal to 1000 S cm⁻¹, less than or equal to 500 S cm⁻¹, less than or equal to 400 S cm⁻¹, less than or equal to 300 S cm⁻¹, less than or equal to 200 S cm⁻¹, less than or equal to 100 S cm⁻¹, or less than or equal to 50 S cm⁻¹. Combinations of these ranges are possible (e.g. less than or equal to less than or equal to 1000 S cm⁻¹ and greater than or equal to 50 S cm⁻¹). Other ranges are also possible.

In some embodiments, the electrically conductive polymer network has a relatively high hydrophilicity. In some embodiments, the hydrophilicity of the electrically conductive polymer network can be measured using a contact angle goniometer. In some embodiments, the electrically conductive polymer network has a relatively low contact angle. In some embodiments, the electrically conductive polymer has a contact angle less than or equal to 10 degrees. In some embodiments, the electrically conductive polymer has a contact angle less than or equal to 50 degrees, less than or equal to 40 degrees, less than or equal to 30 degrees, less than or equal to 20 degrees, less than or equal to 10 degrees, or less than or equal to 5 degrees. In some embodiments, the electrically conductive polymer has a contact angle greater than or equal to 5 degrees, greater than or equal to 10 degrees, greater than or equal to 20 degrees, greater than or equal to 30 degrees, greater than or equal to 40 degrees, or greater than or equal to 50 degrees. Combinations of these ranges are possible (e.g. less than or equal to 50 degrees and greater than or equal to 5 degrees). Other ranges are also possible.

In some embodiments, the electrically conductive polymer and the crosslinker can be cross-linked to form an electrically conductive polymer network at a relatively low temperature. It may be advantageous for the electrically conductive polymer to be capable of cross-linking with the cross-linker at low temperatures as it may allow for the electrically conductive polymer to maintain conductivity through the cross-linking process. In some embodiments, the electrically conductive polymer can be cross-linked with the cross-linker at a temperature less than or equal to 40° C. In some embodiments, the electrically conductive polymer can be cross-linked with the cross-linker at a temperature less than or equal to 140° C., less than or equal to 120° C., less than or equal to 100° C., less than or equal to 80° C., less than or equal to 60° C., less than or equal to 40° C., less than or equal to 25° C., or less than or equal to 20° C. In some embodiments, the electrically conductive polymer can be cross-linked with the cross-linker at a temperature greater than or equal to 20° C., greater than or equal to 25° C., greater than or equal to 40° C., greater than or equal to 60° C., greater than or equal to 80° C., greater than or equal to 100° C., greater than or equal to 120° C., or greater than or equal to 140° C. Combinations of these ranges are also possible (e.g. greater than or equal to 20° C. and less than or equal to 140° C.). Other ranges are also possible.

In some embodiments, the surface comprises a redox active species. In some embodiments, the redox active species comprises PEDOT:PSS. In some embodiments, the redox active species can be any of a myriad of redox active species that is capable of undergoing a redox reaction upon the application of a voltage and/or an electric field at or proximate the surface. Upon the application of a voltage and/or an electric field, the potential difference between the first electrode and the surface may be non-zero. That is, a potential difference exists between the first electrode and the surface thereby inducing a redox reaction between the surface, the cell culture medium, and the first electrode thereby reducing the adhesion of cells attached on the surface. Without wishing to be bound by any particular theory, when the voltage and/or the electric field with respect to the first electrode (e.g. working electrode) is greater than zero, the redox active species can be reduced allowing electrolytes in the cell culture medium to be injected into the surface comprising the redox active species. When the voltage and/or electric field with respect to the first electrode is less than zero, the redox active species is oxidize allowing cationic electrolytes to enter the cell culture medium. The resulting ion flux induces, in some embodiments, a change in cell morphology and reduces the adhesion of cells to the surface. In some embodiments, the reduction and the oxidization of the redox active species can also be associated with a color and/or transparency change in the electrically conductive polymer network.

As an example, in FIG. 2, adhered cells 210A on surface 110B have a relatively flat morphology when electrical system 115 is not active. However, once the electrical system is active, as in FIG. 3, adhered cells 210B appear more rounded due to increased ion flux between surface 110B and cell culture medium 205 resulting in decreased adhesion and cell detachment of the surface as seen with detached cells 315. Surface 110B comprising the redox active species repeatedly oxidizes and reduce in response to the AC voltage and/or field applied between first electrode 120 and surface 110B.

In some embodiments, the surface is configured to reduce the adhesion between the surface and cultured cells on the surface. In some embodiments, the reduction of the adhesion between the surface and cultured cells occurs upon application of an electric field. In some embodiments, the reduction of the adhesion between the surface and cultured cells occurs upon application of a voltage. In some embodiments, the applied the voltage and/or the electric field is an AC voltage and/or a field generated by an AC voltage. In some embodiments, the applied electric field is an AC field. Advantageously, the application of the AC voltage and/or the AC field allows sufficient time for the redox reaction at the surface to be complete and provides sufficient ion flux to thereby induce a morphology change of cells on the surface.

According to some embodiments, as the redox active species reduces and/or oxidizes, the apparent color of the surface may be altered. In some embodiments, the surface is optically transparent. That is, the surface can, in some embodiments, have at least 25% transmission of light at wavelengths anywhere between 350 nm and 700 nm incident normal to the surface through a thickness of at least 0.1 micrometers. In some embodiments, the surface is sufficiently optically transparent to allow for optical microscopy techniques (e.g. cell counting) to be carried out before, during, and/or after the growth and proliferation of cells. In some embodiments, the optical transparency of the surface can change depending on the redox state of the redox active species in the surface.

In some embodiments, the cell culture system comprises an electric system. In certain embodiments, the electric system is configured to apply an alternating electric field (e.g. the electric field) at or proximate to the surface. That is, the electric system, in some embodiments, can generate an electric field such that the surface comprising the redox active species participates in a redox reaction with the first electrode and the cell culture medium when present. The electric system may comprise conductive paths to deliver current and/or apply an electrical potential to the first electrode, the surface, or both. In some embodiments, the first electrode and the surface are electrically coupled via the electric system. In some embodiments, the electric system comprises a power supply, voltage generator, signal generator, electrical switches, and/or other electronic devices configured to deliver, control, and receive electrical signals.

In some embodiments, the electric system is configured to apply an alternating electric field. Advantageously, the application of an alternating electric field, rather than a non-alternating electric field (e.g. a field generated by a DC voltage), provides sufficient time for the redox reaction at the surface to completely oxidize and/or reduce redox active species. The flux of ions that occurs at relatively low frequencies from the cell culture medium into the surface allows the cells to change in morphology and, in some embodiments, detach from the surface. In some embodiments, the electric system is configured to apply an alternating electric field at an frequency capable of altering the morphology of cultured cells. In some embodiments, the electric system is configured to apply an alternating electric field such that the adhesion between the surface and cultured cells on the surface is reduced.

In some embodiments, the AC electric field and/or AC voltage is applied at a frequency by the electric system. Without wishing to be bound by any particular theory, the frequency at which the AC field and/or AC voltage is applied may influence the morphology change of the cells and the corresponding adhesion reduction. That is, relatively high frequency may not provide sufficient time for the redox reaction to complete on the surface. In some embodiments, the AC electric field and/or the AC voltage is applied at a relatively low frequency. In some embodiments, the AC electric field and/or the AC voltage is applied at a frequency greater than or equal to 0.005 Hz, greater than or equal to 0.01 Hz, greater than or equal to 0.02 Hz, greater than or equal to 0.03 Hz, greater than or equal to 0.04 Hz, greater than or equal to 0.05 Hz, greater than or equal to 0.06 Hz, greater than or equal to 0.07 Hz, greater than or equal to 0.08 Hz, greater than or equal to 0.09 Hz, or greater than or equal to 0.1 Hz. In some embodiments, the AC electric field and/or the AC voltage is applied at a frequency less than or equal to 0.1 Hz, less than or equal to 0.09 Hz, less than or equal to 0.08 Hz, less than or equal to 0.07 Hz, less than or equal to 0.06 Hz, less than or equal to 0.05 Hz, less than or equal to 0.04 Hz, less than or equal to 0.03 Hz, less than or equal to 0.02 Hz, less than or equal to 0.01 Hz, or less than or equal to 0.005 Hz. Combinations of these ranges are possible (e.g. less than or equal to 0.1 Hz and greater than or equal to 0.005 Hz). Other ranges are also possible.

In some embodiments, the application of the AC voltage is sufficient to induce the redox reaction but limit damage to cultured cells. Relatively high applied voltages (e.g. greater than or equal to 50V) may damage cells and reduce the cell viability of the cell culture system. Relatively low applied voltages (e.g. less than or equal to 10 V) may provide sufficient redox activity to reduce adhesion with significantly impacting cell viability. In some embodiments, the AC voltage applied between the first electrode and the surface is greater than or equal to 0.1 V, greater than or equal to 0.5 V, greater than or equal to 1 V, greater than or equal to 2 V, greater than or equal to 3 V, greater than or equal to 4 V, greater than or equal to 5 V, greater than or equal to 7.5 V, or greater than or equal to 10 V. In some embodiments, the AC voltage applied between the first electrode and the surface is less than or equal to 10 V, less than or equal to 7.5 V, less than or equal to 5 V, less than or equal to 4 V, less than or equal to 3 V, less than or equal to 2 V, less than or equal to 1 V, less than or equal to 0.5 V, or less than or equal to 0.1 V. Combinations of these ranges are also possible (e.g. less than or equal to 10 V and greater than or equal to 0.1 V). Other ranges are also possible.

In some embodiments, the area of cell coverage on the surface after the application of the voltage and/or the electric field is less than the area of cell coverage on the surface before the application of the voltage and/or electric field. In some embodiments, the area of cell coverage on the surface after the application of the AC voltage for less than or equal to 60 seconds, less than or equal to 50 seconds, less than or equal to 40 seconds, less than or equal to 30 seconds, less than or equal to 20 seconds, or less than or equal to 10 seconds is less than or equal to 50% the area of cell coverage prior to the application of the AC voltage. In some embodiments, the area of cell coverage on the surface after the application of the AC voltage for greater than or equal to 10 seconds, greater than or equal to 20 seconds, greater than or equal to 30 seconds, greater than or equal to 40 seconds, greater than or equal to 50 seconds, or greater than or equal to 60 seconds is less than or equal to 50% the area of cell coverage prior to the application of the AC voltage. Combinations of these ranges are possible (e.g. greater than or equal to 10 seconds and less than or equal to 60 seconds). Other ranges are also possible.

A controllable cell adhesion platform in which cells can be detached from the cell vessel surface using a non-enzymatic strategy in a short time frame was developed. To derive biocompatible, durable, and controllable substrates for cell attachment surfaces, polystyrene was used as a starting material because polystyrene is the material commonly used in cell culture vessels. Polystyrene can be molded using soft lithography techniques to produce substrates which can be used to successfully grow cells.

In some embodiments, the surface comprises a three-dimensionally patterned surface. The three-dimensionally patterned surface, in certain instances, comprises a plurality of protrusions. In some embodiments, the three-dimensionally patterned surface comprises a pattern that reduces the geometric surface area in contact with cells grown on the surface. The reduced geometric surface area reduces the adhesion of the cell to the surface thereby allowing for easier detachment from the surface. In some embodiments, the three-dimensionally patterned surface comprises any of a myriad of three-dimensional patterns including but not limited to protrusions, indentations, peaks, valleys, and/or waves. For example, in FIG. 4, vessel 410 comprises three-dimensionally patterned surface 405. Cultured cells 210A on three-dimensionally patterned surface 405 have a lower contact area with three-dimensionally patterned surface 405 compared to that on a flat surface.

The reduced contact area between the cultured cell and the three-dimensionally patterned surface is directly related to the ease of which cultured may be dislodged and/or detached from the three-dimensionally patterned surface. The ratio of the surface area of each cultured cell contacting the upper geometric surface of the three-dimensionally patterned surface can be expressed according to Equation 1:

$$R = \frac{{NA}_{p}}{{A}_{c}} \qquad Equation 1$$

wherein N is the number of covered protrusions per cell, Ap is the area of the upper geometric surface of the three-dimensionally patterned surface, and Ac is the cell area. The aforementioned ratio must be sufficiently high to ensure adequate contact between the cell and any underlying surface to ensure growth and proliferation, but sufficiently low to allow for cell passage. However, at relatively high ratios, (e.g. R=1) increased adhesion between the cultured cells and any underlying surfaces may result. In some embodiments, the cell culture system has a ratio of the surface area of each cultured cell contacting the upper geometric surface of the three-dimensionally patterned surface less than or equal to 0.7, less than or equal to 0.6, less than or equal to 0.5, less than or equal to 0.4, less than or equal to 0.3, less than or equal to 0.2, or less than or equal to 0.1. In some embodiments, the cell culture system has a ratio of the surface area of each cultured cell contacting the upper geometric surface of the three-dimensionally patterned surface greater than or equal to 0.1, greater than or equal to 0.2, greater than or equal to 0.3, greater than or equal to 0.4, greater than or equal to 0.5, greater than or equal to 0.6, or greater than or equal to 0.7. Combinations of these ranges are possible (e.g. less than or equal to 0.7 and greater than or equal to 0.1). Other ranges are also possible.

In some embodiments, the three-dimensionally patterned surface comprises a plurality of protrusions. In certain instances, the plurality of protrusions comprise a plurality of microposts. According to certain embodiments, the plurality of protrusions have a height sufficient to prevent cultured cells from sagging into the space between each of the plurality of posts. For example, in FIG. 4, three-dimensionally patterned surface comprises a plurality of protrusions 415 with height H1. In some embodiments, the height of the plurality of protrusions on the three-dimensionally patterned surface is greater than or equal to 0.5 micrometers, greater than or equal to 1 micrometers, greater than or equal to 2 micrometers, greater than or equal to 3 micrometers, greater than or equal to 4 micrometers, greater than or equal to 5 micrometers, greater than or equal to 7.5 micrometers, greater than or equal to 10 micrometers, greater than or equal to 15 micrometers, greater than or equal to 25 micrometers, greater than or equal to 50 micrometers. In some embodiments, the height of the plurality of protrusions on the three-dimensionally patterned surface is less than or equal to 50 micrometers, less than or equal to 25 micrometers, less than or equal to 15 micrometers, less than or equal to 10 micrometers, less than or equal to 7.5 micrometers, less than or equal to 5 micrometers, less than or equal to 4 micrometers, less than or equal to 3 micrometers, less than or equal to 2 micrometers, less than or equal to 1 micrometers, or less than or equal to 0.5 micrometers. Combinations of these ranges are also possible (e.g. less than or equal to 50 micrometers and greater than or equal to 0.5 micrometers). Other ranges are also possible.

In some embodiments, the three-dimensionally patterned surface comprises a plurality of protrusions, each protrusion having a width. For example, in FIG. 4, three-dimensionally patterned surface comprises a plurality of protrusions 415 with width W1. The width of the protrusions should be sufficiently large to provide contact area for cultured cells to grow and proliferate but sufficiently small to reduce the contact area between the cell and the surface compared to a flat surface. In some embodiments, the plurality of protrusions each have a width greater than or equal to 1 micrometer, greater than or equal to 2 micrometers, greater than or equal to 3 micrometers, greater than or equal to 4 micrometers, greater than or equal to 5 micrometers, greater than or equal to 7.5 micrometers, greater than or equal to 10 micrometers, or greater than or equal to 15 micrometers. In some embodiments, the plurality of protrusions each have a width less than or equal to 15 micrometers, less than or equal to 10 micrometers, less than or equal to 7.5 micrometers, less than or equal to 5 micrometers, less than or equal to 4 micrometers, less than or equal to 3 micrometers, less than or equal to 2 micrometers, or less than or equal to 1 micrometers. Combinations of these ranges are also possible (e.g. greater than or equal to 1 micrometer and less than or equal to 15 micrometers). Other ranges are also possible.

In some embodiments, the three-dimensionally patterned surface comprises a plurality of protrusions, each protrusion spaced a distance apart. For example, in FIG. 4, three-dimensionally patterned surface comprises a plurality of protrusions 415 each distance D1 apart. The spacing of the protrusions should be sufficiently large to provide contact area for cultured cells to grow and proliferate but sufficiently small to reduce the contact area between the cell and the surface compared to a flat surface. In some embodiments, the plurality of protrusions are each separated by a distance greater than or equal to 1 micrometer, greater than or equal to 2 micrometers, greater than or equal to 3 micrometers, greater than or equal to 4 micrometers, greater than or equal to 5 micrometers, greater than or equal to 7.5 micrometers, greater than or equal to 10 micrometers, or greater than or equal to 15 micrometers. In some embodiments, the plurality of protrusions are each separated by a distance less than or equal to 15 micrometers, less than or equal to 10 micrometers, less than or equal to 7.5 micrometers, less than or equal to 5 micrometers, less than or equal to 4 micrometers, less than or equal to 3 micrometers, less than or equal to 2 micrometers, or less than or equal to 1 micrometers. Combinations of these ranges are also possible (e.g. greater than or equal to 1 micrometer and less than or equal to 15 micrometers). Other ranges are also possible.

In some embodiments, the three-dimensionally patterned surface comprises a plurality of protrusions, each protrusion having an upper geometric surface area. For example, in FIG. 4, three-dimensionally patterned surface comprises a plurality of protrusions 415 with an upper geometric surface area 405. The upper geometric surface area can be modified to increase or decrease the geometric area in contact with growing cells. In some embodiments, the upper geometric surface area of each of the plurality of protrusions is greater than or equal to 5 μm², greater than or equal to 15 μm², greater than or equal to 25 μm², greater than or equal to 50 μm², greater than or equal to 75 μm², greater than or equal to 100 μm², greater than or equal to 125 μm², greater than or equal to 150 μm², or greater than or equal to 175 μm². In some embodiments, the upper geometric surface area of each of the plurality of protrusions is less than or equal to 175 μm², less than or equal to 150 μm², less than or equal to 125 μm², less than or equal to 100 μm², less than or equal to 75 μm², less than or equal to 50 μm², less than or equal to 25 μm², less than or equal to 15 μm², or less than or equal to 5 μm². Combinations of these ranges are also possible (e.g. is less than or equal to 175 μm² and greater than or equal to 5 μm²). Other ranges are also possible.

In some embodiments, the plurality of protrusions comprises a plurality of cylindrical posts. In some embodiments, the cross-section of the plurality of protrusions orthogonal to the height (H1) of the protrusion can be any of myriad of geometric cross-sectional shapes including but not limited to elliptical, triangular, rectangular, pentagonal, hexagonal, and/or octagonal cross-sections. Of course, other cross-section shapes are possible and the cross-sectional shapes may not have perfectly proportional dimensions.

According to some embodiments, the three-dimensionally patterned surface comprises a first material. In some embodiments, the first material comprises polystyrene. Polystyrene is one of the most common materials used in cell culture systems due to its low cost and ease of production. In some embodiments, the first material comprises polystyrene, polycarbonate, and/or glass. The first material, in certain instances, comprises a thermoplastic material that can be molded, shaped, or otherwise deformed at moderate to high temperatures. The first material may be relatively stiff. In some embodiments, the first material has a stiffness of at least 0.5 Gpa, at least 1 Gpa, at least 1.5 Gpa, at least 2 Gpa, at least 3 Gpa, or at least 5 Gpa.

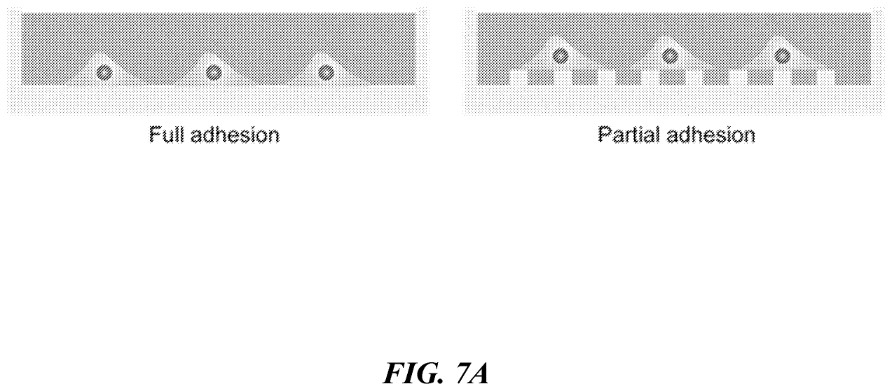

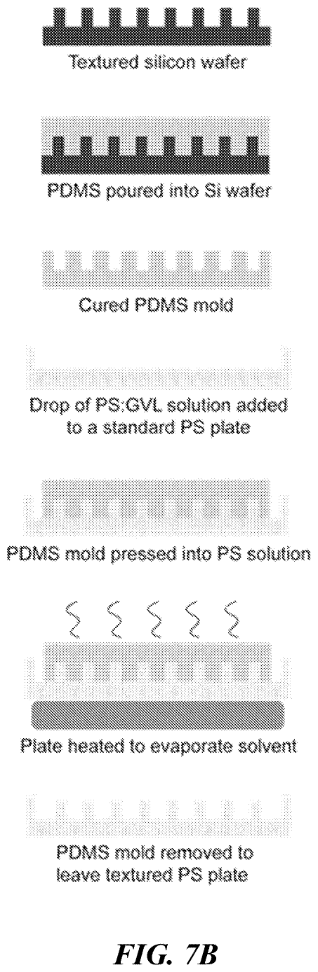

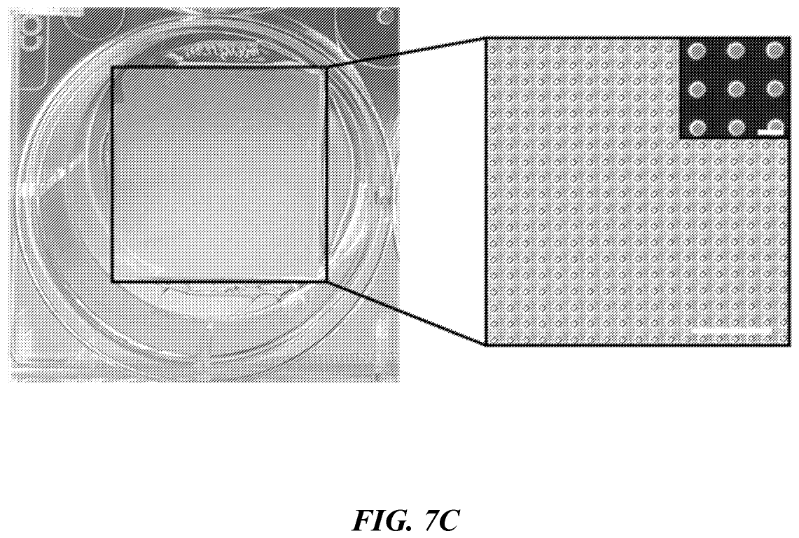

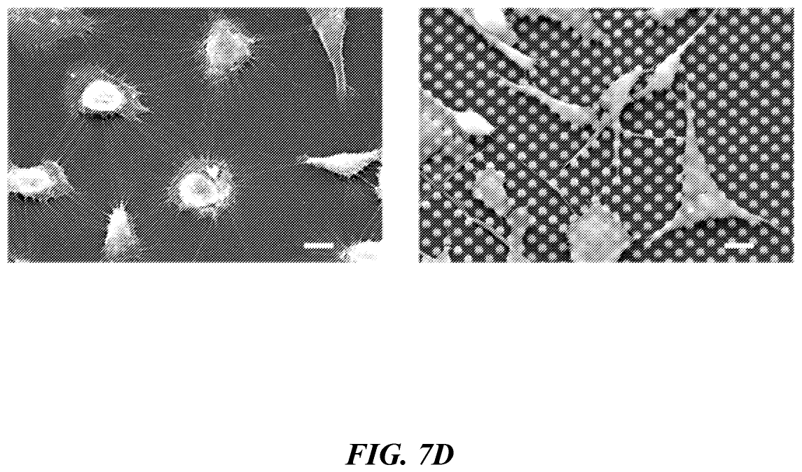

A fabrication protocol was developed for creating micromolded polystyrene surfaces with tunable micron-scale patterns that are optically transparent and biocompatible. Previous studies have shown the use of polystyrene dissolved into gamma-butyrolactone to mold polystyrene surfaces; however, these processes are relatively cumbersome, require high temperatures for evaporation which can degrade the polymer and require large amounts of solvent in order to produce polystyrene surfaces of suitable size for cell culture. With these techniques, a high degree of warping was observed unless PDMS surfaces were coated with a thick enough layer of polystyrene solution (>1 mm), which in turn resulted in extremely long evaporation times of several days. In the demonstrated process, PDMS stamps were employed in a thin layer of added polystyrene:GVL solution drop casted onto commercially available cell culture well-plates. A schematic of the fabrication process is shown in FIG. 7B. This technique requires only a few drops of solution, which evaporate rapidly and at low enough temperatures (80 C) to avoid any degradation of the existing polystyrene plate. Using this method, high-fidelity microposts were produced with features down to 1 um. The surfaces were plasma cleaned after evaporation to remove any chemical contaminants and render the surfaces hydrophilic. Using this platform, surfaces were produced with any microscale pattern desired by first creating a mold using photolithography. The resulting surfaces were optically transparent, hydrophilic, biocompatible, inexpensive, and simple to fabricate.

The three-dimensionally patterned surface can be patterned from the first material, in some embodiments, using a stamping process. A solution comprising the first material as a solute and a suitable solvent capable of dissolving the first material can be cast (e.g. drop casted, spin coated, blade coated) onto a surface of a cell culture vessel (e.g. a petri dish, cell culture flask, bioreactor). A patterned elastomeric stamp (e.g. a PDMS stamp patterned by curing PDMS deposited onto a photolithographically patterned surface) can be stamped onto the cast solution. After heating the surface to remove excess solvent, the PDMS stamp can be removed to yield the three-dimensionally patterned surface. The final pattern on the three-dimensionally patterned surface can be modified by modifying the photolithographic pattern that the uncured PDMS is deposited onto prior to curing.

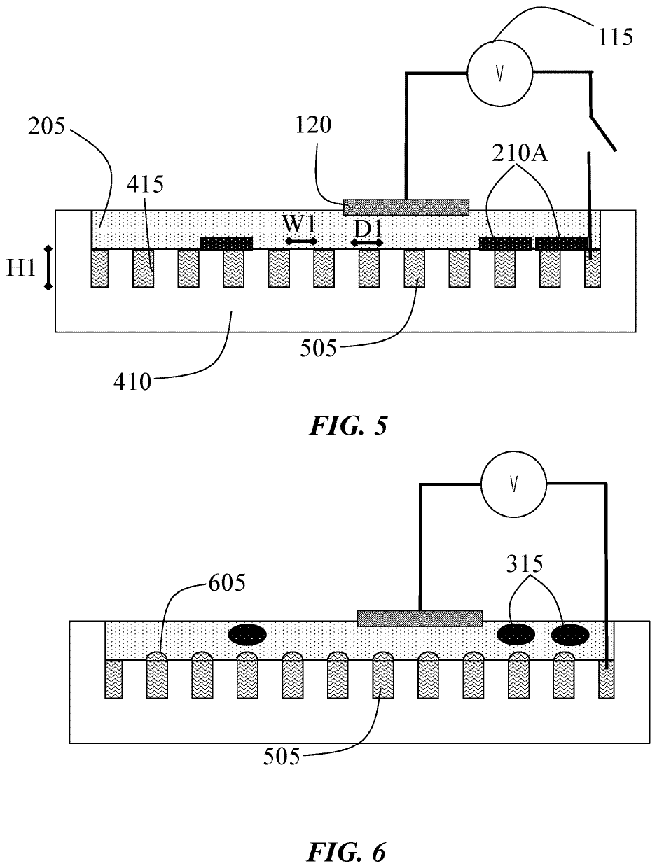

In some embodiments, a second material is dispersed between the plurality of protrusions. In some embodiments, the second material comprises the electrically conductive polymer network. In some embodiments, the second material is configured to exhibit a morphological change upon exposure to the electric field. For example, in FIG. 5, plurality of protrusions 415 comprise the first material and second material 505 is dispersed between plurality of protrusions 415. When electrical system 115 is inactive, cultured cells 210A experienced decreased adhesion on the surfaces of plurality of protrusions 415 due to the lower available surface area compared to a flat surface. When electrical system 115 is active, as in FIG. 6, the voltage is applied to induce morphological change 605 in second material 505 thereby dislodging cells on the plurality of protrusions to produce dislodged cells 315. In some embodiments, the induced redox reaction between the cell culture medium and the electrically conductive polymer network comprising the redox active material swells the electrically conductive polymer network. Since the electrically conductive polymer network is dispersed between the plurality of protrusions comprising the first material, the swelling of the electrically conductive polymer network is at least partially constrained which changes the morphology of the surface of the electrically conductive polymer network. The surface morphology change therefore, in some embodiments, dislodges adhered cells on the surface upon the application of voltage.

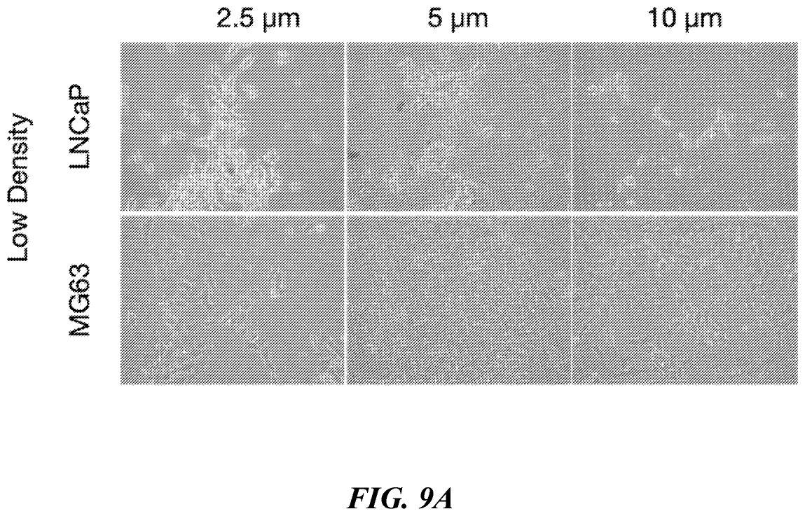

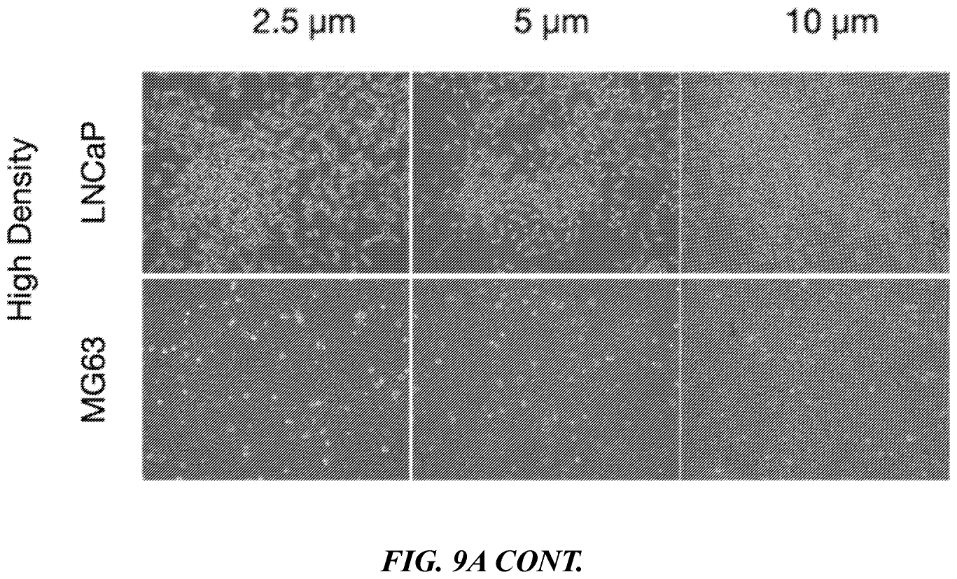

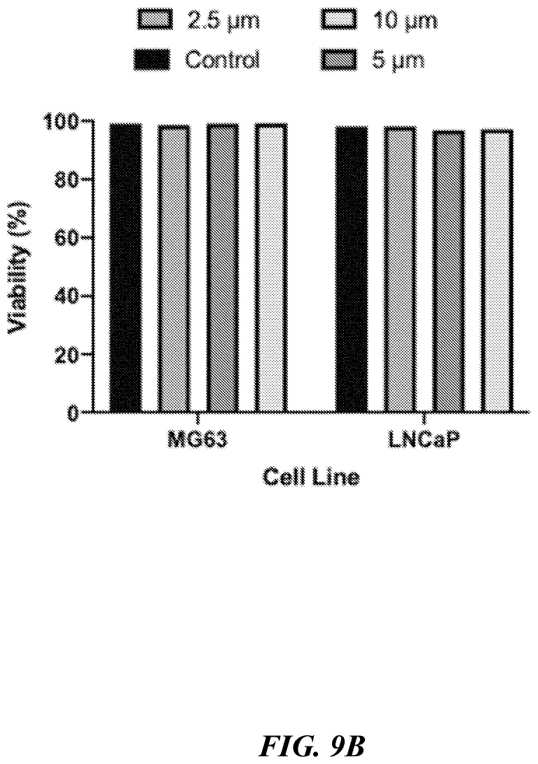

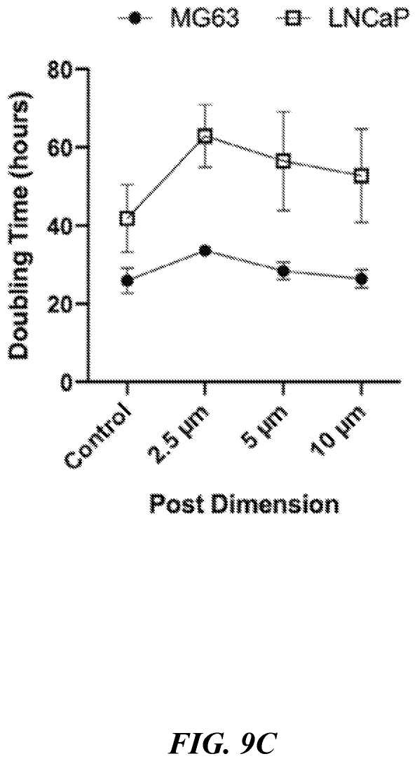

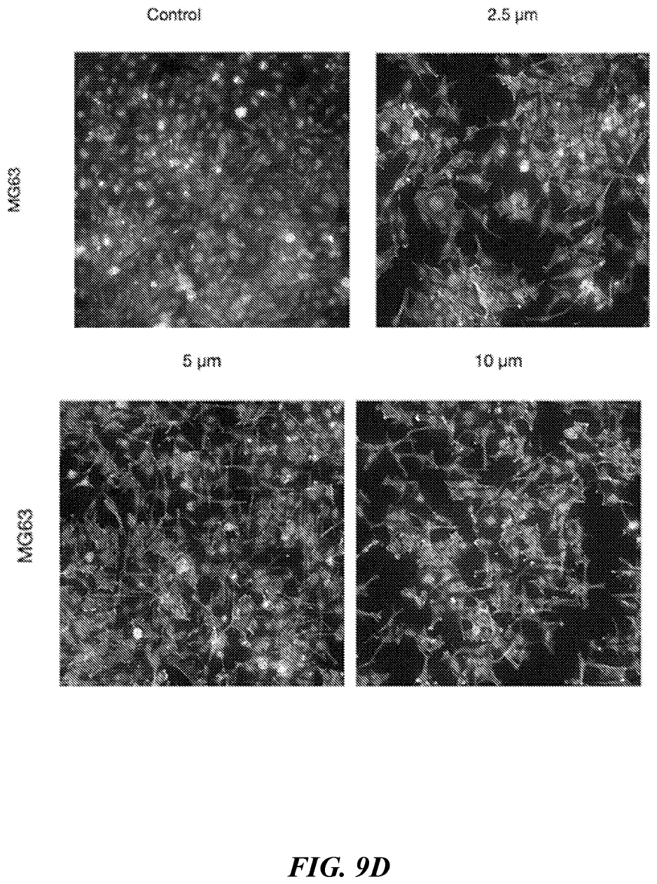

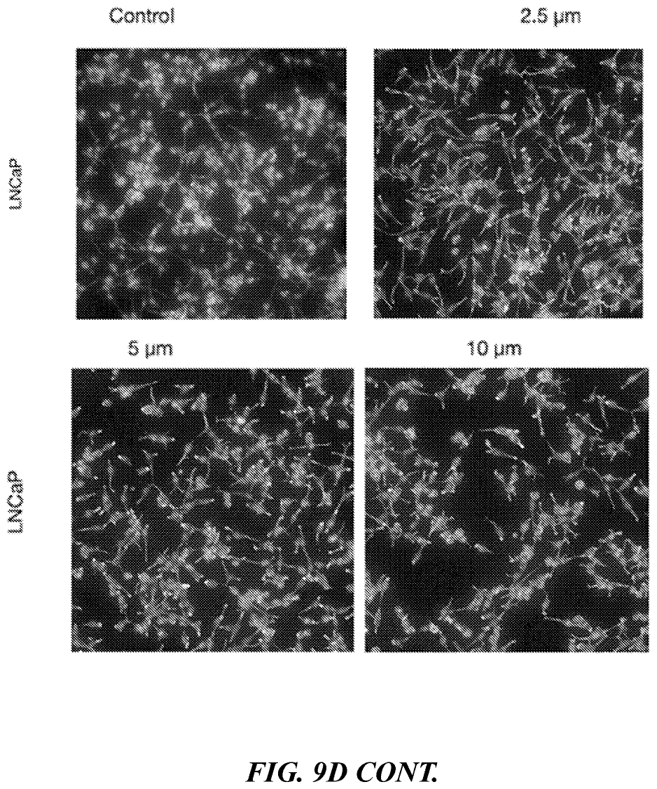

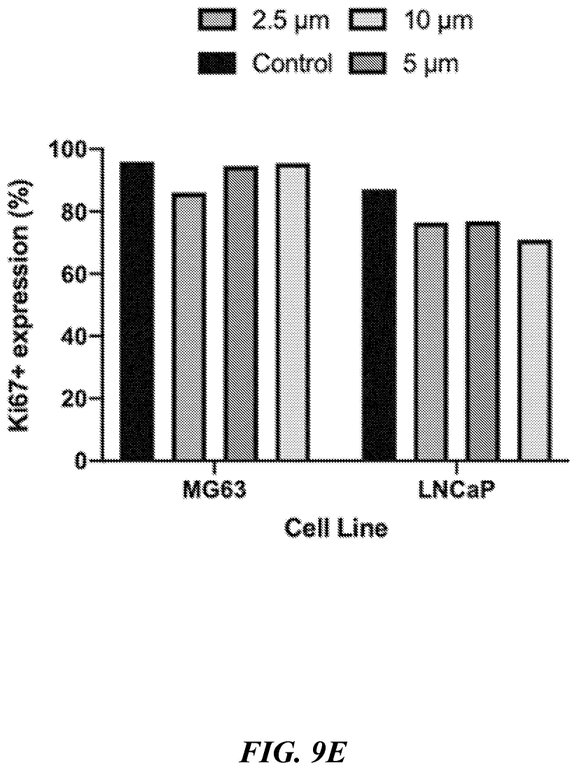

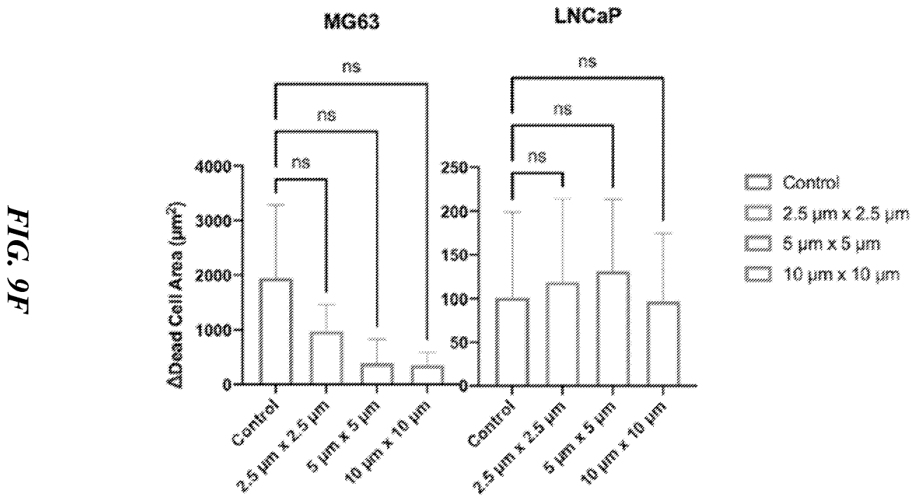

Surface morphology impacts cell adhesion to surfaces. Cells on microtextured surfaces grow and proliferate on the tops of posts, and thus have fewer points of contact and smaller area of contact with the surface compared with flat surfaces, where they grow and spread on the entire surface, as shown in FIG. 7A. The adhesion force of cells to these surfaces was measured using a microfluidic shear platform in order to identify geometries that result in low cell adhesion. Measurements show that cells on microtextured posts have significantly lower adhesion than cells on flat surfaces. In particular, 10 um posts with 10 um spacing showed the most significant decrease in cell adhesion. Importantly, cell viability and proliferation were demonstrated to not be impacted by the microtextures present on these surfaces (FIG. 9B).

Using the microtextured polystyrene surfaces that reduce cell adhesion while maintaining high cell viability and proliferation as a base, a controlled cell adhesion platform was developed wherein an external stimulus can trigger a morphological change of the composite surface, which in turn will initiate cell detachment from the surface. For example, such morphological changes could involve controlled infusion of the soft material or local swelling of the infused material. This is triggered by an external stimulus, such as application of voltage, and will cause cells to detach from the surface in a controlled manner without damaging cells. Previous work has demonstrated ultra-low voltage electrocoalescence by the controlled electrowetting of interfaces. Other recent work has demonstrated the use of liquid-infused poroelastic films to controllably pin and slide droplets by tuning surface wettability with a voltage trigger. Here, a electrowetting processes to develop voltage-triggerable local morphology changes was used.

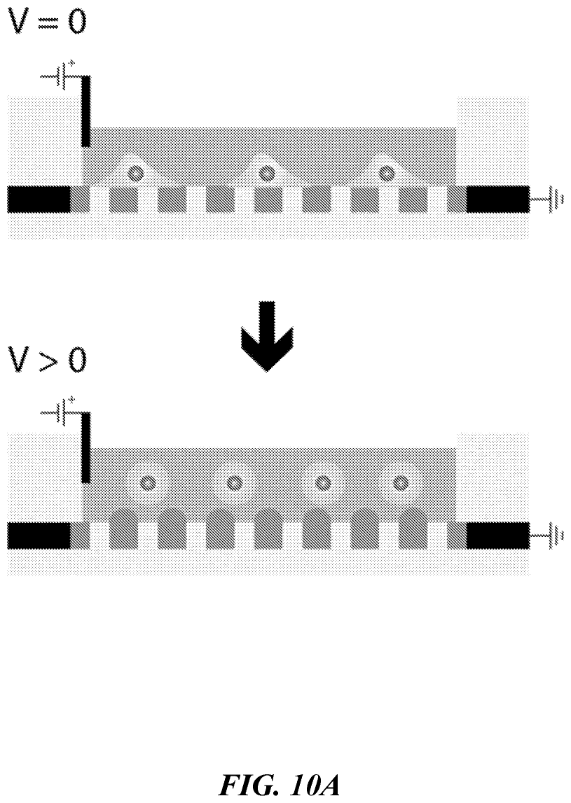

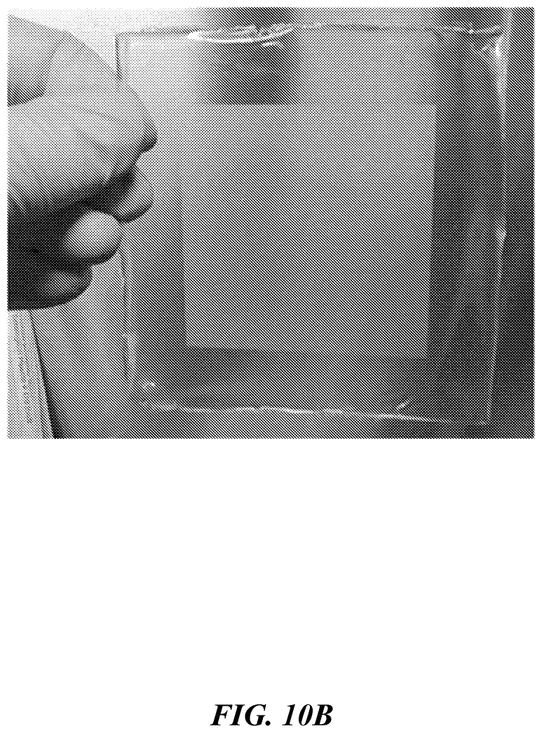

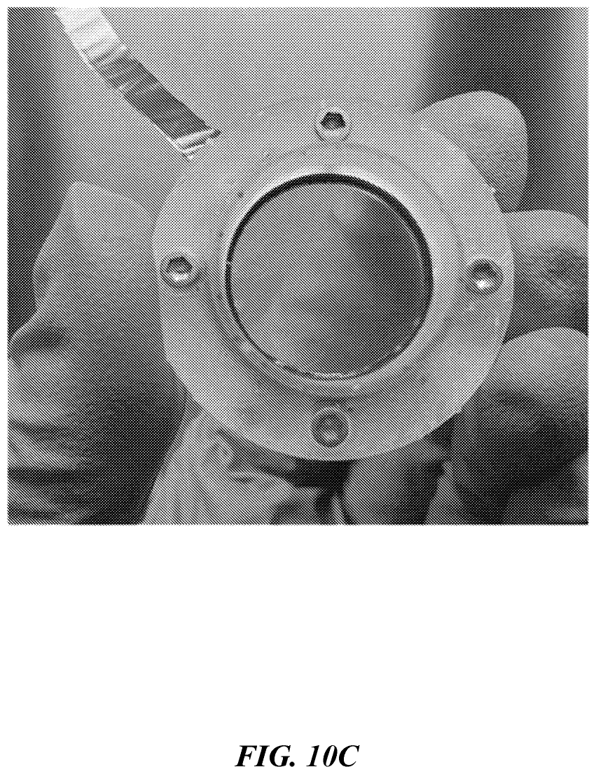

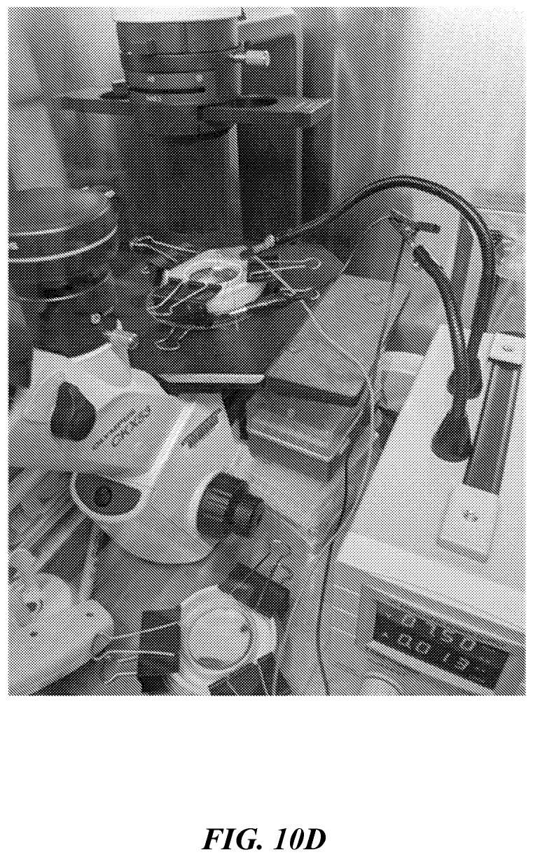

An application of these surfaces using an electroactive polymer infusion was demonstrated. Microtextured polystyrene surfaces with 50 μm post height and 10 μm posts were infused with a solution of 1.3 wt % poly(3,4-ethylenedioxythiphene)-poly(styrenesulfonate) (PEDOT:PSS) in water mixed with 1-5% w/w poly(ethylene glycol) diglycidyl ether, which was spin coated into the polystyrene posts. The surfaces were then cured at room temperature, and then plasma cleaned and sterilized. These composite surfaces, shown in FIG. 10B, were conductive, optically transparent, biocompatible, and robust in an aqueous environment. A prototype was fabricated for applying voltage to these surfaces while cells were grown on them, shown in FIG. 10C. To apply voltage, the copper tape was connected to the conductive surfaces using silver paste. The connected surface was sandwiched into two sides of a 3D printed holder with an O-ring and was tightened with screws or clips to prevent liquid from leaking out. The holder ensures that only the PEDOT:PSS and polystyrene surface comes into contact with the cells but enable the surface to be connected electrically. The voltage is applied between an electrical lead connected to the copper tape and a platinum rod inserted into the cell culture media in the well. The voltage application set-up is shown in FIG. 10D.

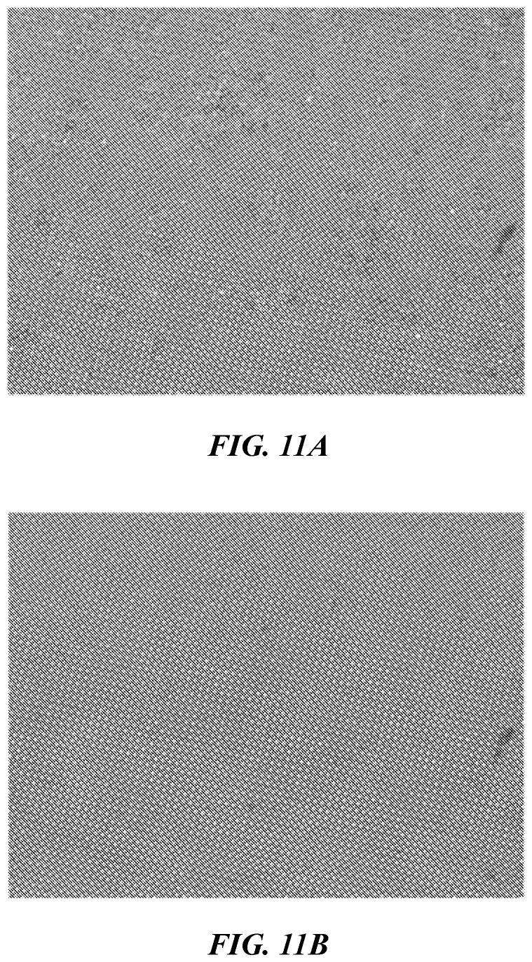

The application of voltage above 3V led to a change in color of the PEDOT surface and to a change in morphology of cells adhered to the surface. After 5 minutes of applying 7.5V, cells were observed to become more rounded, and eventually almost all cells detached from the surface (as shown in FIGS. 11A and 11B).

Accordingly, the cell culture system as described within this disclosure may, in some embodiments, use multiple mechanism to control cell adhesion and/or detachment. In some embodiments, the cell culture system modulates the chemical environment surrounding the cultured cell to reduce adhesion and/or promote detachment. In some embodiments, the cell culture system physically induces the detachment of cells from underlying surfaces. In some embodiments, the cell culture system controls the contact area between the cell and the surface to reduce adhesion thereby facilitating eventual detachment and/or passage.

In some embodiments, the cell culture system comprises the vessel. In some embodiments, the vessel comprises the surface suitable for culturing cells. In some embodiments, the vessel is a petri dish, a bioreactor, a flask such as a flat-sided tissue culture flask, an Erlenmeyer flask, and a spinner flask, or any of myriad of vessels configured to culture cells. In certain instances, the vessel in configured to culture cells in a high throughput manner. In some embodiments, the vessel is capable of being at least partially filled with a cell culture medium. That is, the vessel comprises walls or structures that can contain liquid and/or solid cell culture mediums. In some embodiments, the vessel comprises walls to contain a liquid cell culture medium and prevent contamination of the cell culture medium from external contaminants. In some embodiments, the vessel is configured to passage cells with high throughput. That is, the vessel may comprise at least one inlet and at least one outlet to introduce cell culture medium when necessary and remove passaged cells once detached from the surface.

In some embodiments, the vessel comprises the first electrode. In some embodiments, the vessel comprises the first electrode in electrical communication with the cell culture medium. That is, the vessel can, in some embodiments, be configured such that the first electrode is in contact with the cell culture medium, when present, and the voltage and/or electric field is applied, a redox reaction between the first electrode, the surface, and the cell culture medium occurs. In some embodiments, the vessel comprises the first electrode positioned greater than or equal to 1 mm, greater than or equal to 2 mm, greater than or equal to 3 mm, greater than or equal to 4 mm, greater than or equal to 5 mm, greater than or equal to 7.5 mm or greater than or equal to 10 mm from the surface. In some embodiments, the vessel comprises the first electrode positioned less than or equal to 10 mm, less than or equal to 7.5 mm, less than or equal to 5 mm, less than or equal to 4 mm, less than or equal to 3 mm, less than or equal to 2, or less than or equal to 1 mm from the surface.

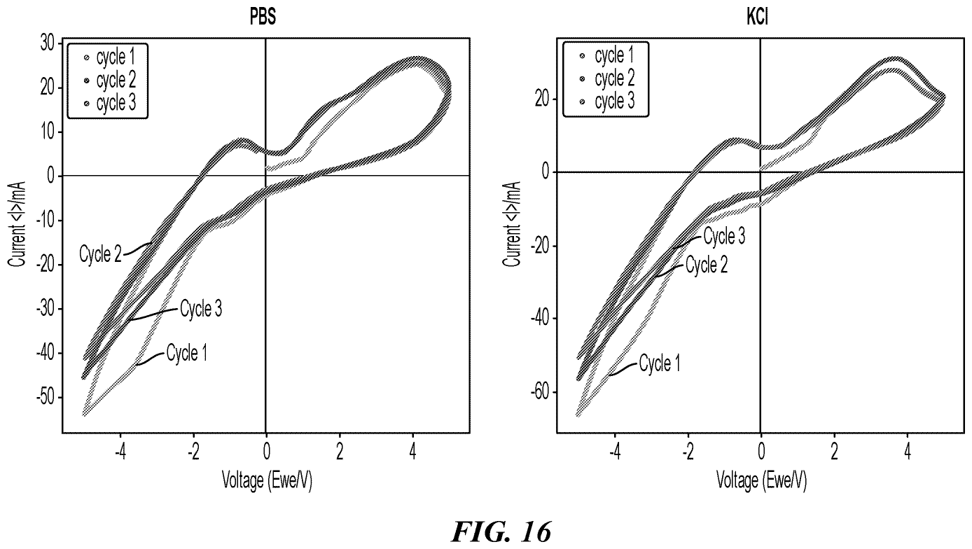

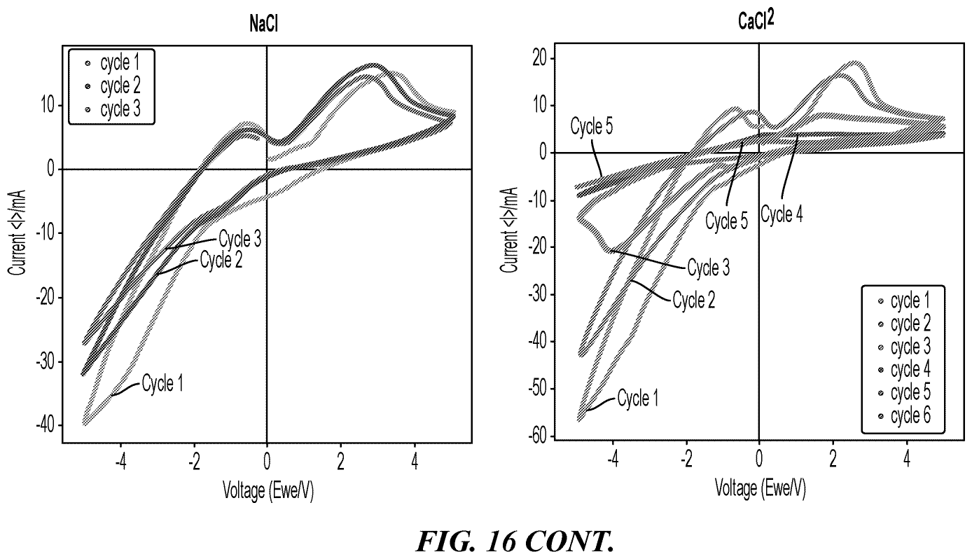

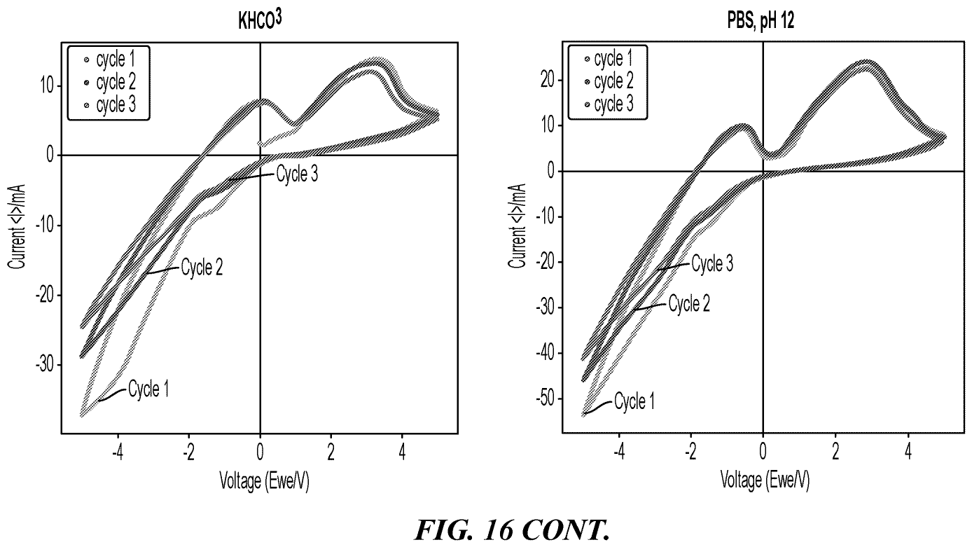

In some embodiments, the cell culture system uses the cell culture medium, when present, as a source of electrolytes that participate in the redox reaction. When the voltage and/or electric field is applied, electrolytes from the cell culture medium enter and/or exit, depending on the polarity of the apply electric field and/or voltage, the surface comprising the electrically conductive polymer network comprising the redox active species. In some embodiments, the cell culture medium, when present, comprises a buffer solution. The buffer solution may have any of a myriad of electrolytes including but not limited to PBS, KCl, NaCl, CaCl₂), and/or KHCO₃ (e.g. FIG. 16).

In some embodiments, the cell culture medium, when present, allows for the first electrode to be in electrochemical communication with the surface. That is, the presence of the cell culture medium allows for the redox reaction between the first electrode, the surface, and the cell culture medium to take place and induce a change in morphology of the cultured cells adhered to the surface. In certain instance, the cell culture medium comprising the buffer solution can be in contact with the first electrode and the surface.

Methods for detaching cells from a surface are generally described. In some embodiments, the method for detaching cells from a surface comprises applying an AC voltage across the first electrode and the surface. In certain embodiments, the method for detaching cells from a surface comprises applying an AC voltage at a frequency capable of altering the morphology of cultured cells. As previously mentioned, the application of an AC voltage advantageously reduces the adhesion between cultured cells and the surface by altering the morphology of adhered cells. In some embodiments, the method for detaching cells from a surface can be performed using the cell culture system.

In some embodiments, the method for detaching cells from the surface further comprises agitating the cell culture medium. The application of a voltage and/or electric field may reduce the adhesion between the cultured cells and the surface, but occasionally, the reduction in adhesion may not be sufficient to dislodge the cell from the surface. In some embodiments, agitating the cell culture medium can dislodge cells that remain on the surface after the application of the voltage. The agitation of the cell culture medium can be performed using any of a myriad of agitation technologies including but not limited stir bars, shakers, and/or bubblers.

In some embodiments, the cell culture system can be capable of culturing any of myriad of cell types. In some embodiments, the cell culture system can selectively affect cell adhesion and/or detachment of cancer cells (e.g. cells from osteosarcoma cell lines or prostate carcinoma cell lines). In some embodiments, the cell culture system can selectively affect cell adhesion and/or detachment of cancer cell lines, patient-derived cultures, primary cells, and/or immortalized different cell lineage normal cells. Detachment and/or controlled adhesion of cells from other cell lines may also be possible.

One aspect of the disclosure herein is a method comprising preparing a polydimethylsiloxane (PDMS) mold, obtaining a polystyrene (PS) cell culture plate, drop casting a thin layer of polystyrene: gamma-valerolactone (PS:GVL) solution on the cell culture plate, pressing the PDMS mold into the PS:GVL solution, heating the plate to remove the solvent, and removing the PDMS mold to produce a textured PS plate. In one embodiment of the disclosed method, the textured PS plate comprises an array of posts. In one embodiment, the posts are 10μ in height and spaced 10μ apart. In one embodiment of the disclosed method, the PDMS mold is produced by photolithography. In one embodiment of the disclosed method, the PDMS mold is produced by additive manufacturing. In one embodiment, the disclosed method further comprises spin-coating the PS plates with poly(3,4-ethylenedioxythiphene)-poly(styrenesulfonate) (PEDOT:PSS) in water mixed with poly(ethylene glycol) diglycidyl ether to produce a PS plate with a conductive surface. In one embodiment, the method further comprises attaching a voltage source to the conductive surface. In one embodiment, the disclosed method further comprises rinsing the surface of the PS plate with a solution to remove contaminants. In one embodiment, the solution comprises plasma. One embodiment are the PS plates produced by the disclosed method.

One aspect of the disclosure herein is an article comprising a cell culture vessel, wherein the cell culture vessel comprises an array of microposts, wherein each micropost has a width of 1-20μ and a height of greater than 5μ, wherein the microposts are separated by 1-10μ. In one embodiment of the disclosed article, the cell culture vessel is comprised of polystyrene. In one embodiment of the disclosed article, the microposts have a cross-section that is circular, square, or triangular. In one embodiment of the disclosed article, the cell culture vessel is a single or multi-well plate. In one embodiment of the disclosed article, each of the microposts further comprise a layered coating. In one embodiment of the disclosed article, the space between the microposts comprises a layered coating. In one embodiment of the disclosed article, the layered coating between the microposts is electrically conductive. In one embodiment the disclosed article further comprises a voltage source attached to the cell culture vessel. In one embodiment the disclosed article further comprises a pump for fluid transport to or from the cell culture vessel. One aspect of the disclosure is a method comprising transferring a cell suspension to the disclosed cell culture vessel to produce adherent cells. In one embodiment, the method further comprises treating the adherent cells by ultra-low voltage to produce detached cells. In one embodiment, the method further comprises culturing the cells to confluence. In one embodiment, the method further comprises collecting the detached cells.

U.S. Provisional Patent Application No. 63/385,235, filed Nov. 29, 2022, and entitled “SURFACES FOR CONTROLLED CELL ADHESION,” is incorporated herein by reference in its entirety for all purposes.

The following examples are intended to illustrate certain embodiments of the present invention, but do not exemplify the full scope of the invention.

### EXAMPLE 1

### Introduction

In this example, poly(3,4-ethylenedioxythiophene) polystyrene sulfonate (PEDOT:PSS) surfaces were demonstrated as a cell culture surface with relatively high biocompatibility, and the ability to decrease cell adhesion on demand. The application of AC voltage to PEDOT:PSS cycled a redox reaction, and ion flux at the cell-surface interface was observed along with reduced cell adhesion which thereby allowed cells to be more easily detached from the surface. It was also observed that, upon application of 5V, the shape of the cells appeared to be more round than that prior to the application of the voltage. When a constant shear force (1.5 nN) was applied, cell detachment increased from 1% to 95% the frequency of the voltage applied was tailored. For this system, the frequency that obtained relatively high amounts of cell detachment was found to be 0.05 Hz. However, at higher frequencies (a frequency of 0.1 Hz and above), the redox reaction was not observed to have time to fully complete, and the cell adhesion was not significantly impacted. Cell viability was not significantly impacted by the use of PEDOT:PSS as a cell culture material or by the application of voltage to the surface, with viability >90%. In all, this example suggests that PEDOT:PSS can be used as a cell culture material that allows for on demand cell detachment via a voltage application.

### Methods and Materials

### Cell Culture

MG-63 osteosarcoma cells were cultured in Roswell Park Memorial Institute (RPMI) 1640 Medium (Gibco, 11835030) supplemented with 10% fetal bovine serum (Sigma, F8317), 100 IU penicillin, and 100 μg/mL streptomycin (Sigma, P4333-100 ML). All cells were maintained at 5% CO₂ and 37° C. Cells were routinely cultured in T-75 flasks and passaged at 80-90% confluence with 0.25% trypsin-EDTA (Gibco, 25200056) for use with experiments.

### Doubling Time and Cell Viability Measurements

Cells harvested at 80-90% confluency were seeded at 2.5e⁵ cells total per well in 6-well plates (corning, 3516). Cells were maintained in standard culture conditions for 48 hours before being trypsinized and measured using the CellDrop Automated Cell Counter (DeNovix) for total cell count and average population viability. Cells were seeded in 6-well plates on standard PS surfaces or PEDOT:PSS and on standard PS after electrical stimulation on PEDOT:PSS at 2.5e⁵ cells per well. After 48 hours, the cells were trypsinized and collected to measure cell viability and total cell counts using the CellDrop Automated Cell Counter. Doubling times were calculated using the standard doubling time equation,

$${T}_{d} = Δ T \frac{ln ( 2 )}{ln ( Δ Q )} ,$$

T_{d}=doubling time, T=cell growth time, Q=population size

### PEDOT:PSS Preparation and Surface Infusion

1.3% PEDOT:PSS (Sigma, 483095) was mixed with 2% v/v PEGDE (Sigma, 475696), and sonicated for 2 minutes. The solution was then used immediately and 1 mL of PEDOT:PSS solution was dropcasted onto a 100 mm polystyrene tissue culture dish (VWR, 10861-680) and allowed to spread across the surface; excess solution was removed. The surfaces were then dried vertically at room temperature for 24 hours, and washed 3 times with DI water.

### Microfluidic Device Preparation

200 μm thick acrylic tape sheets (Nitto Denko Corporation, 5620) were laser cut into 15 mm×40 mm rectangles with 5 mm×25 mm channels cut out. Flat PDMS (Dow Corning Sylgard 184) was cut into 15 mm×40 mm rectangles with two holes punched through to form the channel inlets. The acrylic tape was then adhered to the PEDOT:PSS coated petri dish surface and a PDMS cover was pressed on top. The chips were sterilized with UV for 1 hour and then cells were loaded into channels using a syringe at high density (2×10⁶ cells/mL). Petri dishes were filled with 10 mL of PBS buffer (Gibco) around the chip to prevent any evaporation from the channels. Dishes were then placed in an incubator and cells were grown for 24 hours before performing shear removal experiments.

### Shear Removal Protocol and Analysis

A 60 mL syringe was filled with PBS was connected to 1/16″ OD PTFE tubing (Cole Parmer) and then placed in a syringe pump (Harvard Apparatus). This tubing was inserted into one hole of the microfluidic device, and another tube was inserted to the other hole to remove waste liquid. The devices were placed on an Olympus CX53 inverted microscope at 5× magnification and the microscope was focused on cells within the center of the channel. 0.1 ml/min of flow was applied for 30 seconds to remove any cells that were not adhered to the surface. Shear stress was calculated based on mean cell size to apply 1.5 nN of shear force on all cells. The corresponding flow rate was then applied for 90 seconds, and a video was captured with the Olympus DP22 digital camera. The videos were analyzed using a custom python script to calculate the percent of the surface covered with cells every second throughout the video.

### Voltage Application and Cyclic Voltammetry Measurements

A BioLogic VSP Potentiostat was used to apply sinusoidal and DC voltages to the PEDOT:PSS surface and to measure cyclic voltammetry. The PEDOT:PSS-coated petri dishes were filled with 20 mL of PBS, the counter and reference electrode was connected directly to the dry PEDOT:PSS surface outside of the buffer solution, and the working electrode was connected to a 25 mm×25 mm platinum mesh (Sigma,) which was submerged into the buffer, 5 mm above the PEDOT:PSS surface. All cell imaging measurements were taken under the platinum electrode. The chronoamperometry mode was used to apply constant voltage to the surface, and large amplitude sinusoidal voltammetry mode was used to apply AC voltage.

### Results

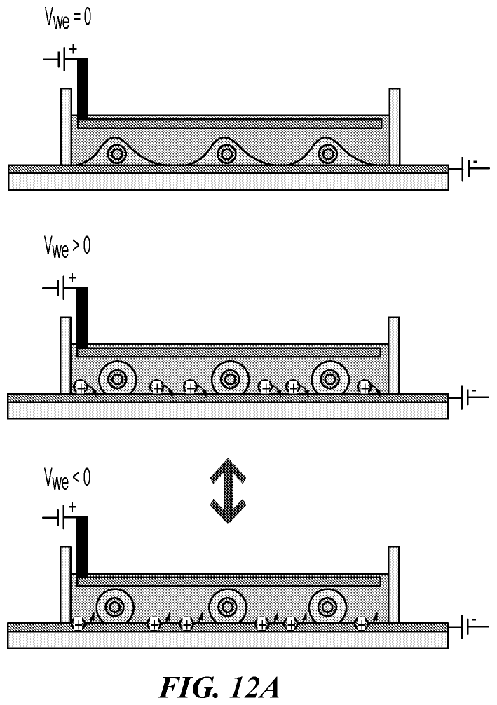

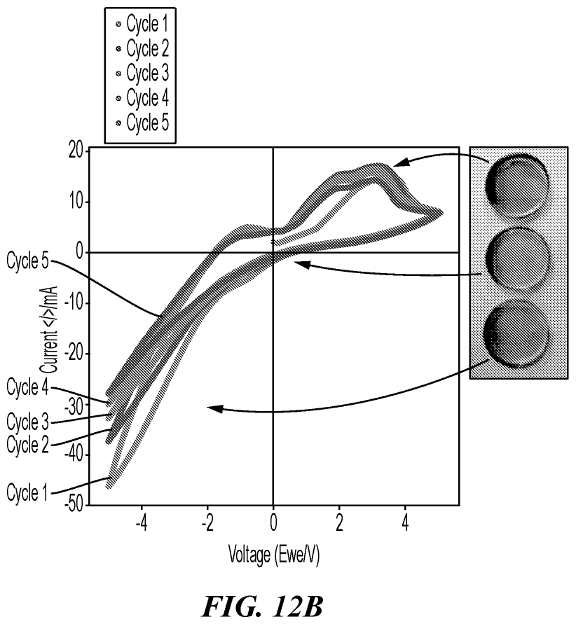

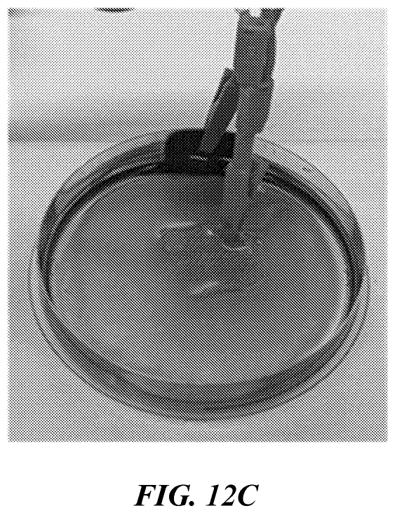

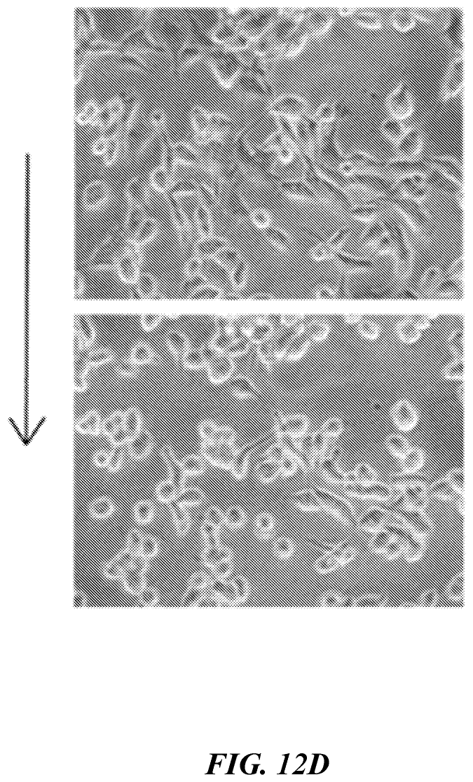

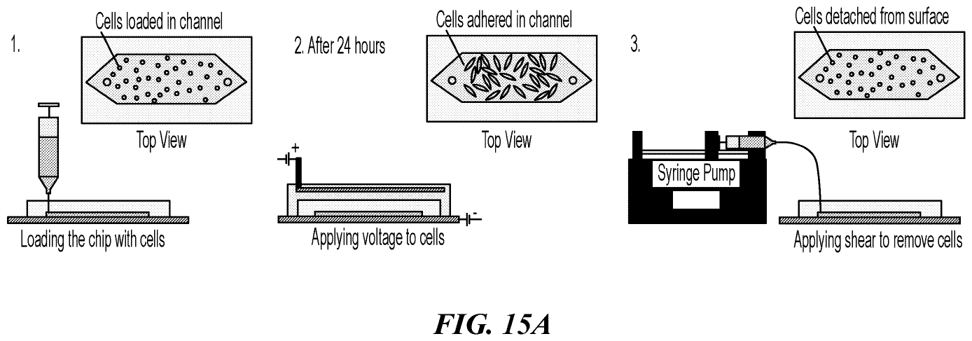

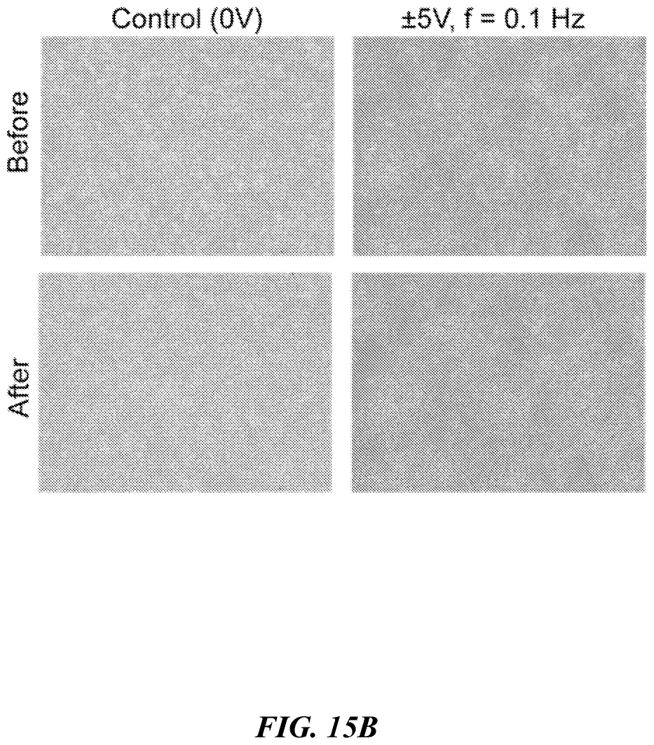

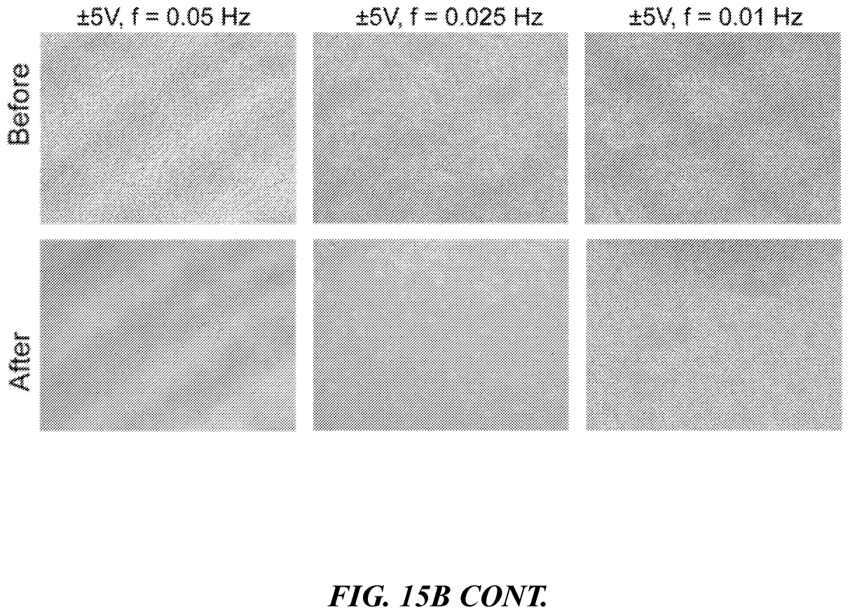

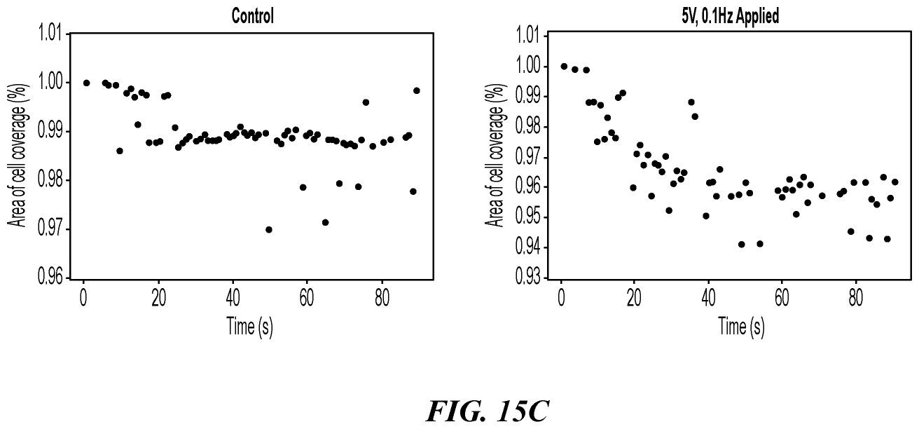

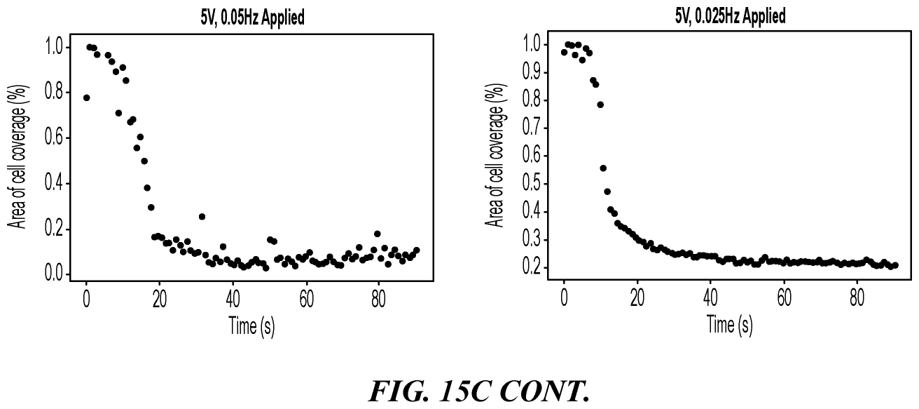

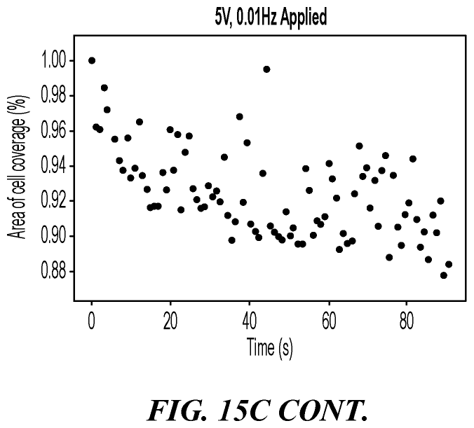

A method for creating PEDOT:PSS films for cell culture using PEGDE as a crosslinker was demonstrated. The films were dropcast directly into polystyrene cell culture dishes and were highly hydrophilic, robust, and conductive. FIG. 12A shows how application of voltage to the surface resulted in cell detachment. When PEDOT:PSS was reduced (V_{we}>0), cations within the electrolyte were injected into the material thereby dedoping the material and altering its color to a dark blue. When the PEDOT:PSS was oxidized (V_{we}<0), the cations were released back into the electrolyte, the material was doped, and it became light blue again. This process led to a change is cell morphology, such that the cells appeared more rounded and easier to detach from the surface. Cyclic voltammetry measurements show the expected redox response. The shape remained consistent cycle after cycle, although there is some shift in the current values over time (FIG. 12B). FIG. 12C shows an image of the electrochemical setup for applying voltage to cells on the PEDOT:PSS surfaces. Applying voltage to cells grown on PEDOT:PSS led to a change in cell morphology, such that cells appeared more rounded after several cycles of the redox reaction (FIG. 15D).

### Mechanism of AC Voltage Impacting Cell Morphology

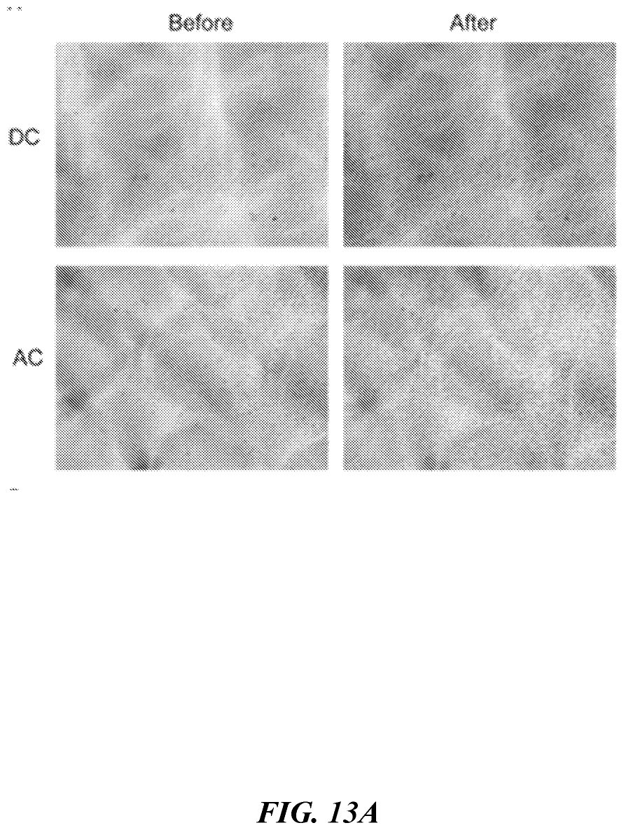

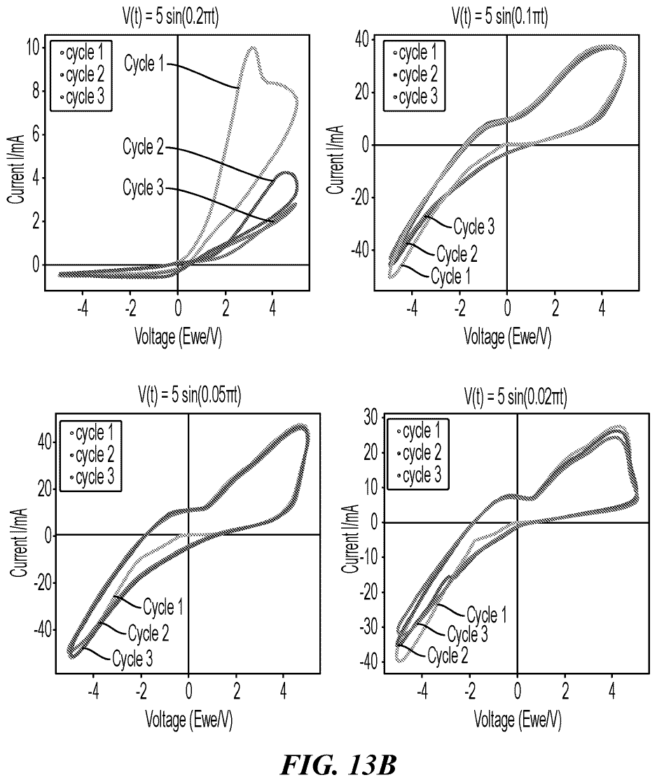

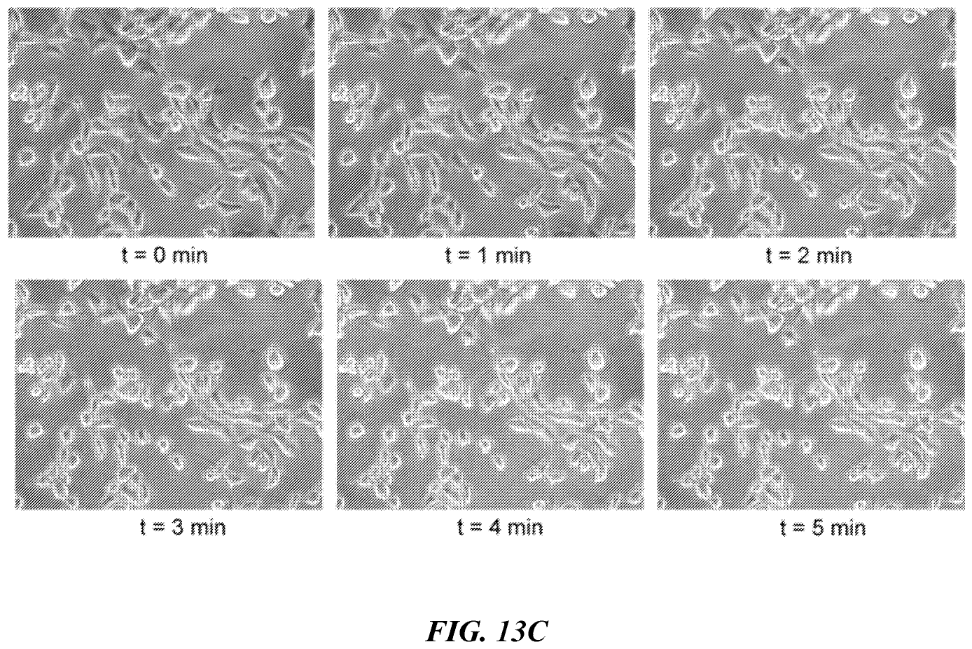

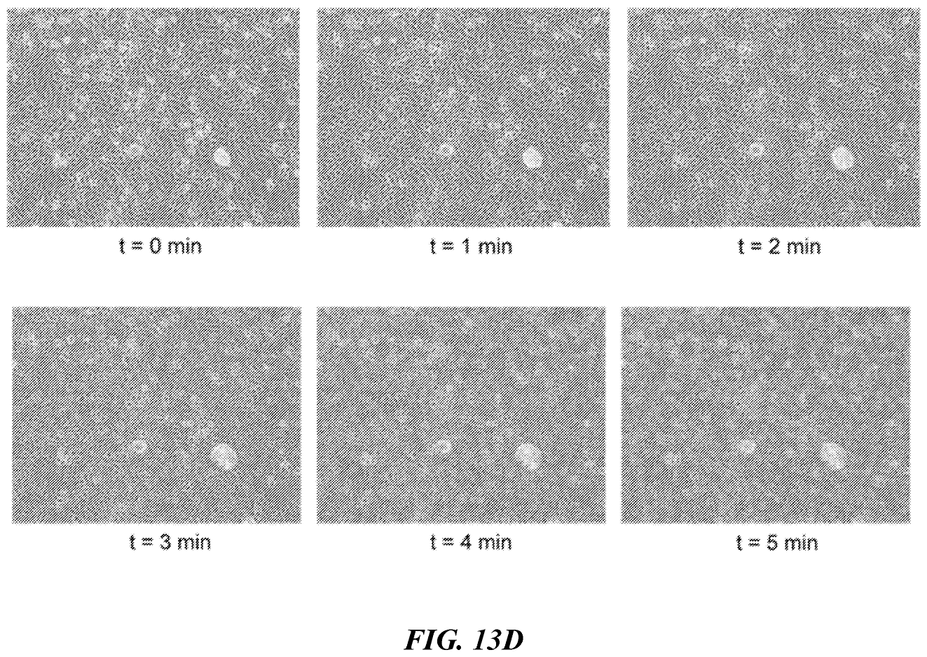

Application of 5V DC resulted in the relatively rapid decline in current applied to the surface within seconds. The cells underwent some migration on the surface in response to the voltage, but no significant morphology change was observed. When 5V AC was applied, the surface experienced continued sinusoidal current over the duration of the voltage application. In contrast with DC voltage, the cells on the surface experienced an observable surface morphology change, with cells becoming more rounded over time (FIG. 13A). The PEDOT:PSS surfaces were sensitive to the frequency of voltage application. When sinusoidal voltage waves were applied, the current peak shifted. At higher frequencies (f>0.1 Hz, the PEDOT:PSS surface was unable to complete the redox reaction across the entire surface, and diminished current values and inconsistent results over repeated cycles were observed. At lower frequencies, the material experienced a current peak at lower voltage values, with a repeatable relationship between voltage and current over time (FIG. 13B). Upon the application of 5V with a frequency of 0.05 Hz, cells were observed to become more rounded within minutes (FIG. 13C). Similarly, when cells were exposed to a more acidic pH, with elevated levels of H⁺ ions in solution, they experienced a similar morphology change, with cells becoming visibly more round as well (FIG. 13D). Taken together, these results suggest that the impact of AC voltage is likely due to the impact of current which causes ion flux at the surface from the redox reaction that PEDOT:PSS undergoes, rather than the voltage resulting in a surface charge or electrical field.

### Cell Viability and Doubling Time

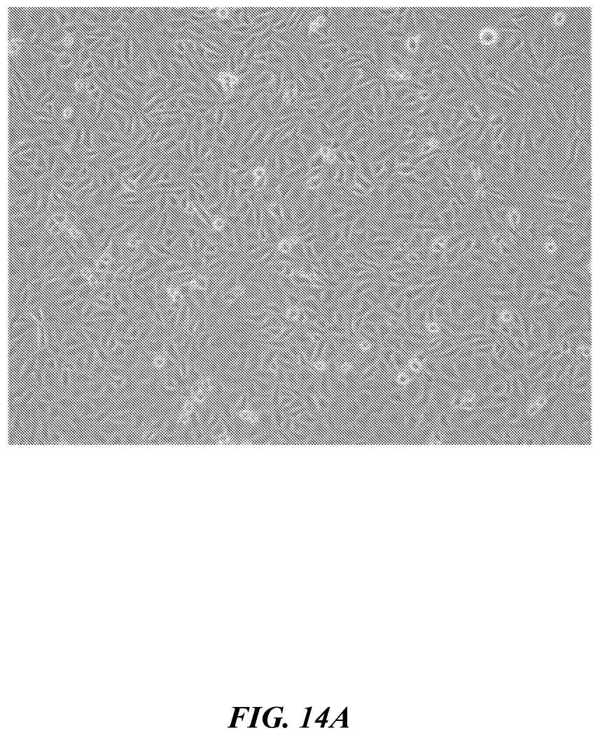

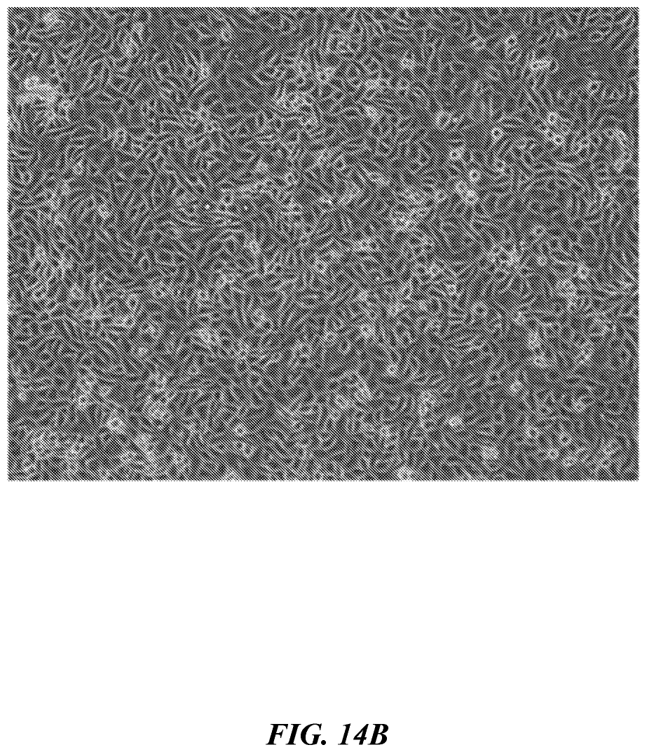

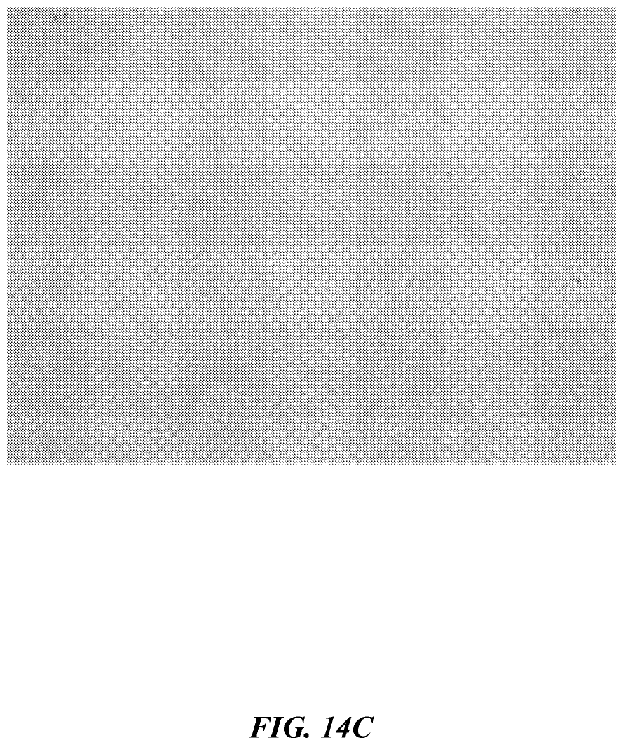

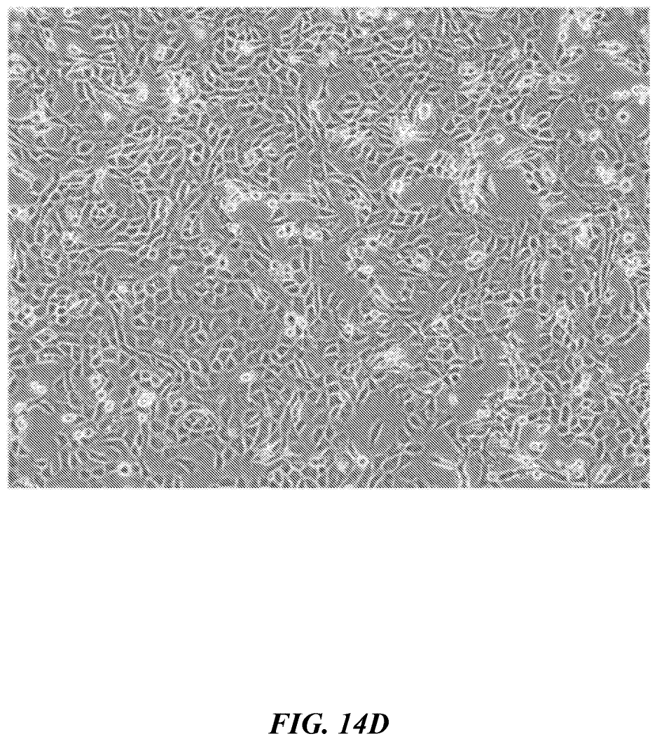

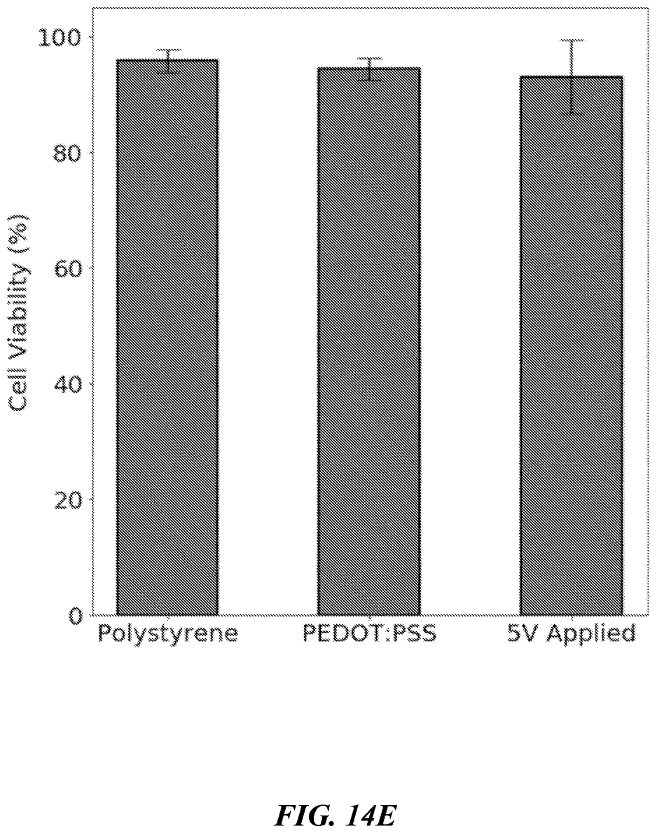

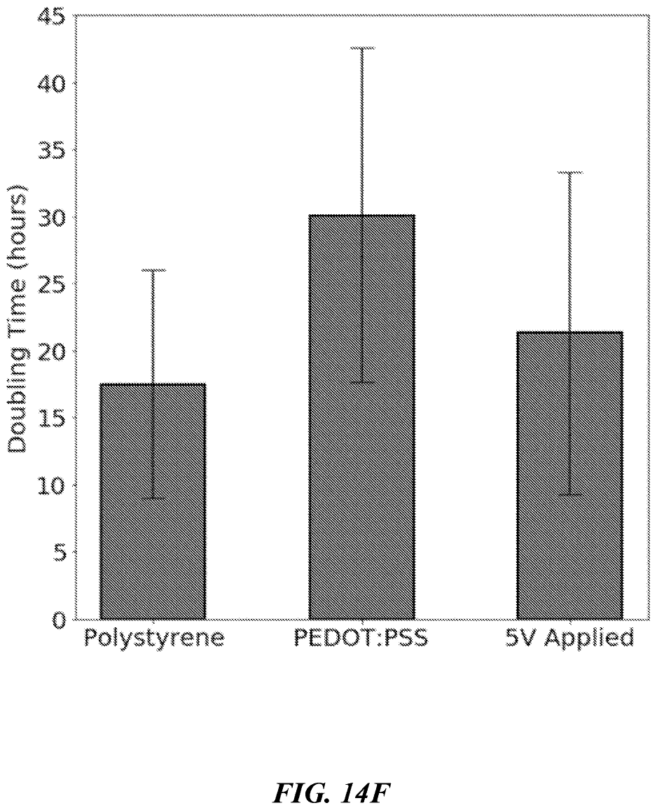

MG63 human osteosarcoma cells were grown on the PEDOT:PSS surfaces to evaluate their biocompatibility. Comparison of brightfield images of these cells on regular polystyrene surfaces (FIG. 14A) and on the PEDOT:PSS surfaces (FIG. 14B) shows that cell morphology was not impacted by the material. Application of 5V AC led to a change in cell morphology, causing cells to be more rounded (FIG. 14C). A sinusoidal voltage wave with 5V amplitude and frequency of 0.05 Hz was applied to MG63 cells grown on PEDOT:PSS for 5 minutes (15 cycles). After voltage application, cells were trypsinized and re-seeded on a polystyrene surface (cell density=2.5e⁵) and grown for 48 hours (FIG. 14D). Cells resumed their normal morphology, indicating that the effect of voltage application, which causes the cells to become rounded, is temporary. Cell viability on PEDOT:PSS surfaces and on PEDOT:PSS surfaces after application of AC voltage (5V amplitude and frequency of 0.05 Hz) were not significantly different than on standard polystyrene surfaces. The mean cell viability measured on polystyrene was 96%, on PEDOT:PSS was 94% and immediately after voltage application was 93% (FIG. 3E). Cell doubling time was increased when cells were grown on PEDOT:PSS, from a mean time of 17.5 hours on standard polystyrene to a mean of 30.1 hours on PEDOT:PSS. However, after voltage was applied and cells were re-seeded on standard polystyrene, mean cell doubling time was 21.3 hours.

### Using Shear to Measure Cell Detachment

A microfluidic device was used to evaluate the adhesion strength of cells grown on PEDOT:PSS as sinusoidal voltages were applied over a range of frequencies. Cells were grown within microfluidic channels for 24 hours. A voltage was then applied to cells for 5 minutes and then immediately after, a constant shear force was applied for 90 seconds to measure the amount of cells detached from the surface (FIG. 15A). Videos were taken during the shear application (FIG. 15B), and the percent of cell coverage was determined at each time point using a python script (FIG. 15C). The PEDOT:PSS surfaces showed the strongest adhesion, higher than standard polystyrene cell culture plates, with 1% of cells detached after 90 seconds of 1.5 nN shear application. The impact of the application of AC voltage on cell adhesion was dependent on voltage frequency. At the highest frequency, 0.1 Hz, 4% of cells were detached, while at the lowest frequency, 0.01 Hz, 11% of cells were detached. Intermediate frequencies were optimal for cell detachment, with 80% of cells removed at 0.025 Hz, and 95% of cells removed at 0.05 Hz.

PEDOT:PSS crosslinked with PEGDE was hydrophilic (contact angle<10°), transparent and biocompatible, making it a promising material for cell culture. Results demonstrated that PEDOT:PSS was compatible with a human cell line, with comparable cell viability to standard cell culture vessels. Cell doubling time increased when cells were grown on PEDOT:PSS, however, this impact was transient and limited to directly growing cells on the PEDOT:PSS, rather than in contact with the material. Cell doubling time values stabilized when cells were grown on polystyrene again. The application of voltage to cells using the PEDOT:PSS surfaces had no significant impact on long term viability or cell doubling time.

A redox reaction was observed upon application of a voltage to PEDOT:PSS. Without wishing to be bound by any particular theory, when the PEDOT was reduced (a positive voltage was applied to the working electrode), cations from the electrolyte (PBS) intercalated into the PEDOT:PSS film, thereby de-doping it and resulting in swelling of the film. When the PEDOT is oxidized, the cations may be expelled from the material, thereby doping the material. Cyclic voltammetry measurements of PEDOT:PSS surfaces with different buffers used as an electrolyte supported that PEDOT:PSS can intercalate a wide range of biologically relevant cations, including Na⁺, K⁺, Ca²⁺, and H⁺. Cycling the redox reaction of PEDOT:PSS via sinusoidal voltage led to continued ion flux at the cell-surface interface. The influence of different cations within PBS on cell adhesion was explored by exposing cells to high concentrations of different cations with solutions of 1M NaCl, 1M KCl and 0.01M HCl. Exposing cells to each of these solutions for 5 minutes and then performing shear cell adhesion measurements revealed that only HCl exposure resulted in a cell morphology change and an increase in cell detachment. Thus, without wishing to be bound by any particular theory, H⁺ flux at the cell-surface interface may be at least partially responsible for the increase in cell detachment observed as voltage is applied to the PEDOT:PSS surface. The lack of cell response to DC voltage application suggests that surface charge or electrical fields alone are not responsible for the cell morphology changes that are observed.

### Conclusion

A process based on cycling an electrochemical reaction on PEDOT:PSS surfaces was demonstrated. and allowed for cell adhesion to be significantly reduced, such that cells can be mechanically detached from the surface without the use of trypsin. Applying voltage with the appropriate frequency to PEDOT:PSS surfaces may offer a fast, simple platform for on-demand cell detachment without the need for trypsin, and could benefit researchers that work delicate cell types. The demonstrated process could also impact the development of cancer treatments, specifically in developing and manufacturing cell-based therapeutics and personalized medicine by enabling primary cells from tumors to be more easily cultured in labs, possible treatments to tested in vitro, and by simplifying the process for culturing cells used in personalized treatments.

### Example 2

### Introduction

In this example, a novel method to precisely design and manufacture tailored micro-structured PS surfaces for controlling cell adhesion is presented. Cylindrical posts molded on PS surfaces with varied densities (1:1 spacing and diameter) for their use as cell culture surfaces were assessed. These post surfaces modulated the level of adhesion of cells to the surface exclusively on the basis of surface morphology. By using PS microscale posts on the order of cell size, cell adhesion strength to the surface can be decreased, while the cell growth properties of a normal surface were maintained. To ensure that there is no impact on cell growth by microtextures, cell viability, proliferation, and apoptosis in two well-characterized cell lines, MG-63 (osteosarcoma cell line) and LNCaP (prostate carcinoma cell line) were determined. Additionally, cell image analysis was performed to quantify cell morphology changes, and a microfluidic-based method was developed to measure the levels of cell adhesion to these surfaces. As a result, these microtextured surfaces allow for cell adhesion with reduced surface contact, thus facilitating easier cell detachment. Such surfaces can inform the development of fluidic or other active cell detachment methods based on lower cell detachment forces and would not require the use of trypsin.

### Methods and Materials

### Cell Culture

MG-63 osteosarcoma cells and LNCaP clone FGC prostate cancer cells (LNCaP) were cultured in Roswell Park Memorial Institute (RPMI) 1640 Medium (Gibco, 11835030) supplemented with 10% fetal bovine serum (Sigma, F8317), 500 ng mL-1 Fungizone (Cytiva, SV30078.01), 20 μg mL-1 gentamicin (Thermo, 15710064), 100 IU penicillin, and 100 μgmL-1 streptomycin (Sigma, P4333-100ML). All cells were maintained at 5% CO2 and 37° C. Cells were routinely cultured in T-75 flasks and passaged at 80-90% confluence with 0.25% trypsin-EDTA (Gibco, 25200056) for use in experiments.

The MG-63 and LNCaP cell lines were both used to observe the impact of the microtextured PS surfaces on morphology, proliferation, variability, doubling times, and shear force testing. The MG-63 cell line was exclusively used for image segmentation and downstream morphological quantification as it grows in even monolayers. LNCaP cell images were not amenable to this analysis due to their tendency to form multilayered aggregates.

### Doubling Time and Cell Viability Measurements

Cells harvested at 80-90% confluency were seeded at 2×105 cells total per well in 6-well plates (Corning, 3516) with or without molded microtextured surfaces. Cells were maintained in standard culture conditions for 4 days before being trypsinized and measured using the Vi-CELL XR Cell Viability Analyzer (Beckman Coulter, 731196) for total cell count and average population viability. Cells were seeded in 6-well plates either with or without microtextured posts at 2×105 cells per well. After 4 days, the cells were trypsinized and collected to measure cell viability and total live cell counts using the Vi-CELL XR Cell Viability Analyzer. Doubling times were calculated using the standard doubling time equation:

$${T}_{d} = \frac{Δ T * ln ( 2 )}{ln ( Δ Q )} \qquad Equation 2$$

where T_{d}=doubling time, T=cell growth time, and Q=population size.

### Fluorescence Imaging of Cells Cultured on Microtextured Surfaces

Imaging experiments were performed in 6-well plates (corning, 3516) with or without molded microtextured surfaces and seeded with either 4×105 MG-63 or LNCaP cells total per well. Cells were allowed 48 h to adhere before being washed 1× with PBS (Thermos, 10010023) and fixed with 4% paraformaldehyde (PFA) for 15 min at room temperature. Cells were washed 3× with ice-cold PBS and then permeabilized using 0.1% Triton-X 100 solution in PBS for 10 min at room temperature. Cells were again washed 3× using ice-cold PBS. Wash buffer was prepared as 2% fetal bovine serum (Sigma, F8317) and 5 μm EDTA (Sigma, 20-158) in PBS and kept on ice. Recombinant anti-Ki67 antibody (clone SP6) conjugated to AlexaFluor488 (Abcam, ab281847) was diluted 1:50 in wash buffer. Alexa Fluor 647 Phalloidin (Invitrogen, A22287) was prepared according to the manufacturer's protocol and added to each well with an anti-Ki67 staining solution to completely cover the cells. Cells were incubated in the dark, on ice for 30 min before staining solution was aspirated and cells were washed 3× with ice-cold wash buffer. Hoechst 33342 staining solution (Invitrogen, H3570) was diluted to a final concentration of 5 μgmL-1 in PBS, and sufficient volume to cover cells was added to each well. Cells were incubated for 10 min at room temperature protected from light before immediate imaging on the Operetta CLS High Content Analysis System (Perkin Elmer). At least n=15 000 cells were incorporated into each measurement. All image analysis was performed using the Harmony high-content and imaging analysis software.

### Incucyte Cell Death Assay

Cell death over time was assessed using the Incucyte S3 System to image cells stained with the Incucyte Cytotox Green Dye (Sartorius, 4633) at the manufacturer's recommended 1:4000 dilution in complete media. MG-63 or LNCaP cells were seeded in 24-well plates (Fisher Scientific, 10177380) with or without molded microtextured surfaces and seeded with 2×104 cells per well. Images were captured once every 4 h for 7 days to track cell death over time as measured by the area of green fluorescent signal in μm2. An aggregate end-point analysis of total dead cell area using the Incucyte analysis software was performed to quantify the percent change from seeding.

### EdU Assay Flow Cytometry Analysis

All thymidine analog 5-ethynyl-2′deoxyuridine (EdU) labeling assays were performed with an EdU Staining Proliferation kit (iFluor 488) (Abcam, ab219801) with kit reagents prepared according to the manufacturer's protocol. Cells were prepared for experiments using 6-well plates as previously described. EdU solution was added to cells at a final concentration of m and incubated for 24 h. On the day of experimentation, cells were trypsinized, washed 2× with ice-cold wash buffer, and stained with Zombie Red fixable viability dye (Biolegend, 423 109) at a 1:500 dilution in wash buffer for 30 min on ice protected from light. The staining solution was washed out by centrifugation and further washed 3× with ice-cold wash buffer before fixing it with 4% PFA for 15 min at room temperature. Cells were centrifuged and washed 3× with ice cold wash buffer and then permeabilized using 0.1% Triton-X 100 solution in PBS for 10 min at room temperature. Cells were again centrifuged and washed 3× with ice-cold wash buffer. The EdU reaction solution was prepared and added according to the manufacturer's protocol. Reaction solutions were prepared fresh and used no later than 15 min after preparation. Cells were analyzed on a CytoFLEX S flow cytometer (Beckman Coulter, CO9764) with iFluor 488 fluorescent signal recorded and measured in FlowJo (Treestar). Dead cells labeled with Zombie Red fixable viability stain prior to fixation were excluded from the analysis.

### Microtextured Surface Fabrication

Micropost designs were prepared using AutoCAD, and silicon wafers were etched to 5 m depth using standard photolithography techniques. In brief, 1 μm of AZ3312 photoresist (AZ Electronic Materials) was spin-coated onto silicon wafers, a direct writing maskless aligner (Heidelberg MLA150) was used to crosslink the post-design, and then wafers were developed using propylene glycol methyl ether acetate (PGMEA). The posts were then etched to 5 μm depth using a deep reactive ion etcher (DRIE). Etched silicon wafers were then functionalized using 1H,1H,2H,2H-perfluorododecyltrichlorosilane (Sigma, 729 965) by depositing 50 μL onto a glass slide and placing alongside a wafer under vacuum for 2 h, thereby making the wafers hydrophobic to prevent PDMS from sticking. PDMS (Sylgard 184, Dow Chemical) was mixed according to package directions, poured onto the silicon wafer, degassed, and cured at 75° C. for 1 h. The PDMS mold was then peeled away from the wafer and cut, using razor blades, into smaller pieces, 8 mm×8 mm, for 24-well plate use.

25% PS:GVL solution was made by dissolving PS beads (Sigma) into the solvent GVL (Sigma, 918 660). PS beads were added to glass jars containing the solvent at 25% w/w and sonicated for 72 h to fully dissolve. 300 μL drops of PS:GVL solution were added to each well in standard PS cell culture 6-well or 24-well plates (Corning Costar), and PDMS mold pieces were pressed micropost side down into the drops. Well plates were placed on hot plates inside a fume hood at 75° C. for 8 h to evaporate all the solvent. PDMS molds were peeled off to expose molded PS micropost surfaces inside the wells of the cell culture plate. Well plates were cleaned with oxygen plasma for 4 min in a plasma cleaner (Glow), and then washed with 70% ethanol and placed inside a sterile environment to dry. Flat control surfaces were prepared using the same protocol but with flat PDMS.

### Scanning Electron Microscopy

Cells were cultured on microtextured flat PS laminated to glass coverslips until reaching 80% confluence. Coverslips were washed with 1 mL PBS warmed to room temperature. PBS was aspirated and 0.5 mL of 2.5% glutaraldehyde fixative solution (VWR, 16120) was added to each coverslip. Cells were fixed at room temperature for 1 h before being washed 3× using deionized water for 2 min each. Osmium tetroxide (Sigma, 75632) fixation was performed using a 1% solution at room temperature for 1 h. Dehydration was performed using a graded ethanol series (25%, 50%, 75%, 95%, and 100%). Coverslips were submerged once for 5 min each, except in the 100% ethanol where coverslips were submerged 3× for 10 min each. Samples were dried for 15 min at room temperature in a 1:1 hexamethyldisilazane (HDMS) (Sigma, 440 191)-ethanol solution. The solution was aspirated and cells were then dried with 100% HDMS at room temperature 2× for 15 min. Excess liquid was wicked with filter paper before leaving cells to dry in a fume hood overnight. Samples were coated with 5 nm of Au/Pd using an EMS Q150T ES coater. They were imaged using a JEOL 6610LV Low Vacuum Scanning Electron Microscope at 20 kV using the high vacuum detector.

### Measurement of Cell Size and Shape

MG63 cells were grown on microtextured and flat surfaces and imaged using the Operetta CLS High Content Analysis System (Perkin Elmer) after 48 h. A custom Python image analysis program was then used to segment each image into individual cells. This program was used to analyze the morphological properties of each individual cell such as cell area, perimeter, and length. The circularity of the cells, C, was also calculated to determine how much the cells were spreading on the surfaces

$$C = \frac{4 π A}{{P}^{2}} \qquad Equation 3$$

where A=area of the cell and P=perimeter of the cell.

### Microfluidic Chip Fabrication

Microtextured PDMS was molded into cell culture Petri dishes following the procedures described above and then oxygen plasma was treated for 4 min. 200 μm thick acrylic tape sheets (Nitto) were laser cut into 15 mm×40 mmrectangles with 5 mm×25 mm channels cut out. Flat PDMS was cut into 15 mm×40 mmrectangles with two holes punched through to form the channel inlets. The acrylic tape was then adhered to the microtextured surface and a PDMS cover was placed on top. The chips were sterilized with UV for 1 h and then cells were loaded into channels using a syringe at high density (2×106 cells mL-1). Petri dishes were filled with 10 mL of PBS buffer (Gibco) around the chip to prevent any evaporation from the channels. Dishes were then placed in an incubator and cells were grown for 24 h before performing shear removal experiments.

### Shear Removal Protocol and Analysis

A 60 mL syringe was filled with PBS (Gibco), connected to “ 1/16” tubing, and then placed in a syringe pump (Harvard Apparatus). This tubing was inserted into one hole of the microfluidic device, and another tube was inserted into the other hole to remove waste liquid. The devices were placed on an inverted microscope at 5× magnification and the microscope was focused on cells within the center of the channel. 0.1 mLmin-1 of flow was applied for 30 s to remove any cells that were not adhered to the surface. Shear stress was calculated for each post size based on cell shape analysis results to apply 1.5 nN of shear force on all cells. The corresponding flow rate was then applied for 90 s, and a video was captured as the shear was applied. The videos were analyzed using a Python script to calculate the percent of the surface covered with cells every second throughout the video, as described in the Supporting Information.

### Statistical Analysis and Plots

All numerical analysis and plots generated unless otherwise noted were produced in GraphPad Prism 9.00. Statistical significance in experiments was determined by a one-way analysis of variance (ANOVA) test. Results are expressed as means with standard deviation from n=3 replicates for experiments.

### Results

### Fabrication of Microtextured Polystyrene Surfaces

The goal of this study was to evaluate how microtextures impact cell adhesion strength by reducing cell-surface contact area, as illustrated in FIG. 7A. A new procedure was established to create transparent, biocompatible microtextured PS cell culture surfaces, shown in FIG. 7B, which allows for the evaluation of cell-surface adhesion strength. In this method, a PDMS stamp was applied into a thin layer of dissolved PS solution drop-cast onto commercially available cell culture well-plates. This technique requires only 300 μL of solution per well, which evaporates in less time (<8 h) compared with the days it previously took and at low enough temperatures (80° C.) to avoid any degradation of the existing PS plate. Gamma-valerolactone (GVL) is an environmentally friendly solvent that costs about $100 L−1. Using this method, high-fidelity microposts with features down to 1 μm were produced. Optical and scanning electron microscopy (SEM) imaging were used to observe the posts from a top view in an off-the-shelf 6-well plate to ensure the microtexture quality (FIG. 7C). The surfaces were plasma cleaned after evaporation to remove any chemical contaminants and render the surfaces hydrophilic. Once the silicon wafer masters were produced using standard photolithography techniques, they could be used indefinitely to make PDMS molds for this process. In addition, PDMS molds were reusable as well. The process (FIG. 7B) took ≈10 min of hands-on work per wafer, 1 h of waiting for PDMS mold preparation, and 8 h of waiting for PS surface preparation. The resulting surfaces were optically transparent, hydrophilic, inexpensive, and quick to fabricate.

MG-63 and LNCaP cancer cell lines were selected for growth tests to understand whether these microtextured PS surfaces are biocompatible. Cells were grown on round microposts with post heights of 5 μm, and post diameters and spacings of 2.5, 5, and 10 μm. In each case, the post diameter was kept the same as the post spacing. Using high-magnification SEM, cells were observed to be able to grow on top of the microposts, as expected. Cells grown on the top of microposts showed reduced surface area contact (FIG. 7D) in comparison to the cells grown on the flat control surfaces (FIG. 7D). Additionally, the morphology of the cells grown on the micropost surfaces was clearly elongated compared with the flat controls. Thus, we next examined the biological and morphological properties of this cell-surface interaction in order to elucidate these observations.

### Impact of Microtexture on Doubling Time and Viability

To investigate if microtextured PS surfaces are biocompatible for tissue culturing, we performed cell viability assays using the MG-63 and LNCaP cell lines. It is a well-known phenomenon that cells in a new environment may undergo stressors that impact their ability to proliferate. To test this, we prepared two different cell seeding densities for doubling time analysis (FIG. 9A,C). According to the cell confluency calculation, cells appear to adhere well and populated quickly 48 h after seeding on all post sizes compared to the flat control (FIG. 9C). To investigate whether these microtextured post surfaces could truly affect cell proliferation, standard doubling time calculations and viability assessments were carried out. The calculated growth assay doubling times showed no statistically significant differences between all microtexture post dimensions and the flat control surface for both MG63 and LNCaP cell lines (FIG. 9C). Additionally, cell viability assessment showed high (>95%) viability in cells across all microtextured surfaces and the flat control (FIG. 9B), indicating that our microtextured surface fabrication process is biocompatible and can be applied to tissue culturing experiments. Taken together, these results suggested the rapid adaptability of established human cancer cell lines to be cultured on microtextured PS surfaces without negatively impacting growth.

### Molecular Level Evaluation of Cell Turnover

Cell death is a naturally occurring process during cell culturing as there is always some degree of cell turnover between cell passages. Since the cell population viability accounts for both cell proliferation and cell death, the idea that the various microtextured posts could lead to different cell turnover rates by using molecular biomarkers of cell division and apoptosis was explored. Cell cycle phases across all microtextures were evaluated by using complementary flow cytometry staining and immunofluorescence imaging assays. Through two different markers, flow-based EdU was used to detect cells in the S phase and anti-Ki67 staining to visualize and measure cells outside the quiescent GO phase.

The actin cytoskeleton was visualized using phalloidin to highlight the previously observed changes in cell morphology on microtextured surfaces. The MG-63 cell line in particular displays a more exaggerated flattened and stretched shape leading to a thinner or “compressed” cytoskeleton. This contrasts with its normally heterogenous oval-shaped and thick spindle-like cells. LNCaP cells already display a thin, spindle-like morphology that did not drastically change on the microtextured surfaces. However, a diminished ability in this cell line to aggregate in large clusters was observed. Anti-Ki67 staining co-staining was conducted to quantify proliferation as measured by the fraction of positively stained cells from the total population as detected using fluorescent nuclear counterstaining. According to fluorescence imaging (FIG. 9D), differences in the fraction of cells staining positive for Ki67 (shown in green) were not statistically significant across all microtextures and flat controls, suggesting similar cell division rates.

Total DNA replication was measured through the incorporation of EdU into cellular DNA after its addition to the cell culture medium. Total EdU content was detected using a green fluorescent azide and quantified by flow cytometry to determine the fraction of proliferative EdU+ cells. Quantification performed at either the protein or DNA levels did not demonstrate significant differences in cell proliferation in MG-63 or LNCaP cells after culturing on microtextured surfaces of different post dimensions compared to flat PS surfaces. Cells generally maintained 85-90%+ proliferation in all conditions, as is expected for established cancer cell lines. LNCaP cells displayed an average 5-10% lesser proliferative population than MG-63, which is also expected and reflective of the longer doubling times in this cell line.

Finally, to assess the cell death rate during cells grown on microtextured surfaces, Live Cell IncuCyte assays were performed by using the Cytotox Green Reagent to stain for the apoptotic biomarker Annexin V. The percent change in the fluorescent area, indicating cell death, from the initial time-point to 7 days in culture (FIG. 9E) shows no statistically significant difference in cell death between all microtextures and the flat control. These concordant results for both cell proliferation and cell death assays agree with the findings that microtextured PS surfaces do not affect cell cycle and doubling times compared to flat PS surfaces and may therefore be suitable for routine cell culture applications.

### Impact of Microtexture on Cell Morphology

The results of the cell shape analysis corresponded well with the previous SEM image observations. A significant difference in the total cell area of the cells grown on the flat control surfaces compared with the cells grown on the micropost surfaces was observed, with a mean size of 343.4 μm2 on flat surfaces, and 122.8, 147.7, and 145.8 μm2 on the 2.5, 5, and 10 μm post surfaces, respectively. This shows that the micropost surfaces lead to a much smaller cell area compared with flat control surfaces. Similarly, there was a significant difference in the circularity of the cells on the flat control surfaces and micropost surfaces, as well as a slight difference between the cells on the 2.5 μm surfaces and 10 μm surfaces, indicating that the cells were more elongated on the micropost surfaces, with the greatest elongation on the 2.5 and 5 μm surfaces. There remains a linear relationship between the cell length and cell area over all of the surfaces. The slope of this is steeper in the post surfaces (m_{2.5}=0.20, m₅=0.20, and m₁₀=0.19) than the flat control surfaces (m_{flat}=0.10), providing further indication that the cells were more elongated on the micropost surfaces than the flat control surfaces.

A new image-based method of determining the ratio of cell-surface contact area was used in order to compare the levels of cell adhesion between the different surfaces, the contact ratio method. The number of posts per cell in each cell image was counted for a total of 200 cells. The number of internal posts (posts fully covered by the cell) was then counted, as well as external posts (posts partially covered by the cell). On the post surfaces, the number of posts per cell as a function of cell area was fairly linear, demonstrating a greater number of posts per cell for the smaller posts, as expected. Finally, the ratio of the surface area of each cell contacting the effective surface area of the post surface was analyzed using Equation 1. For a flat surface, this ratio is 1 for each cell. The post contact ratio was similar for each of the micropost surfaces (R_{2.5}=0.58, R₅=0.6, and R₁₀=0.5), but smallest on the 10 μm post surfaces, where the average ratio was 0.5. This indicates that the adhesion strength of the cells grown on the micropost surfaces could be as low as half of the adhesion strength of the cells grown on the flat control surface.

### Measurement of Cell Adhesion Strength

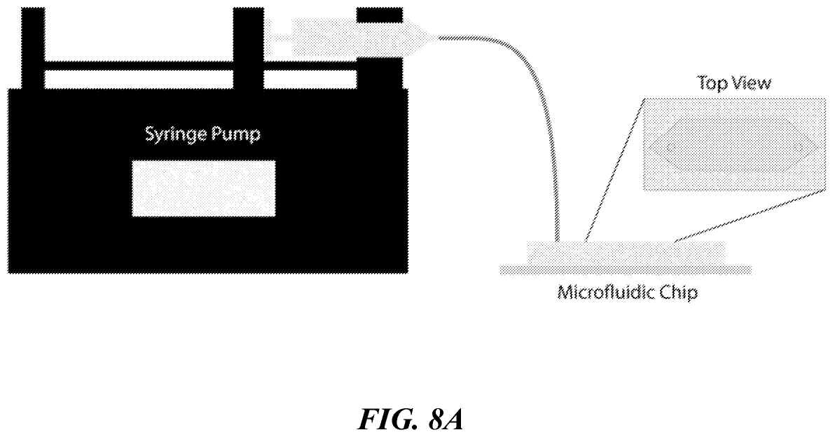

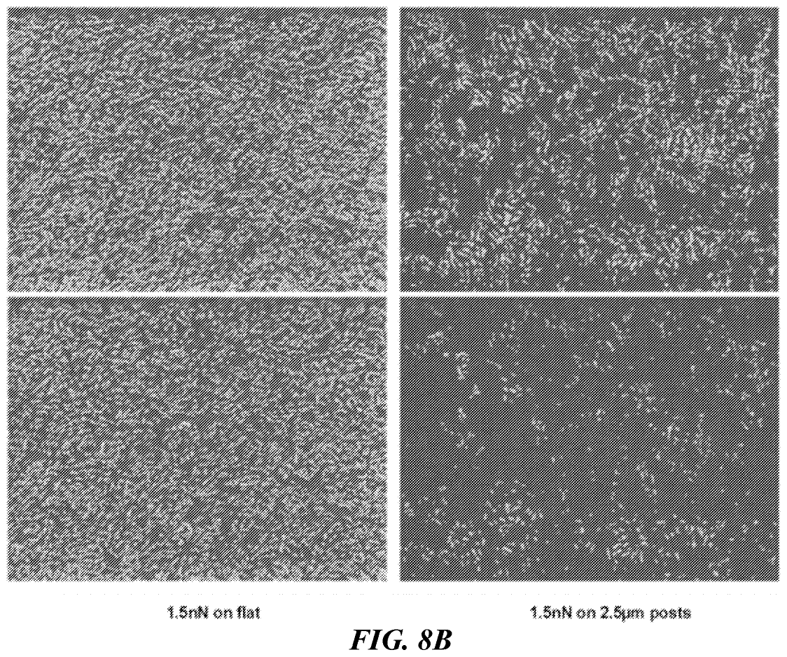

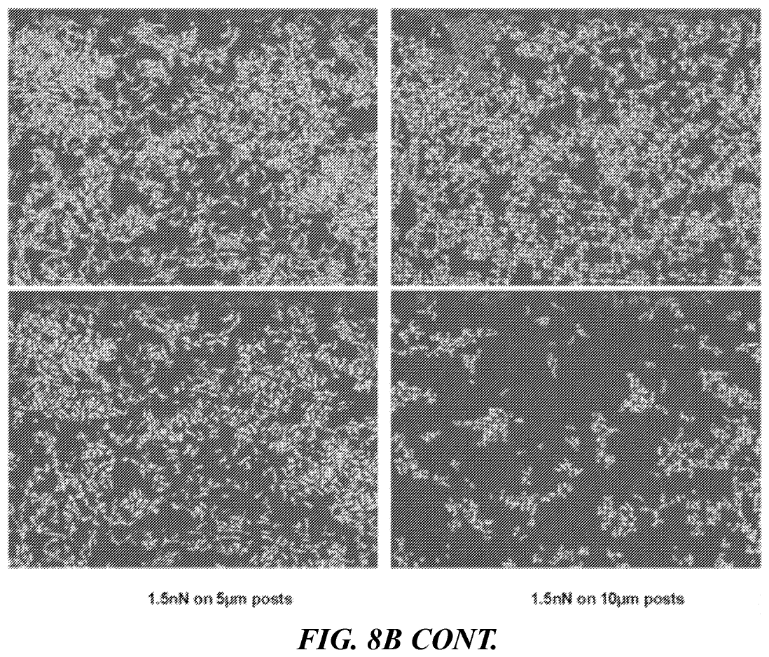

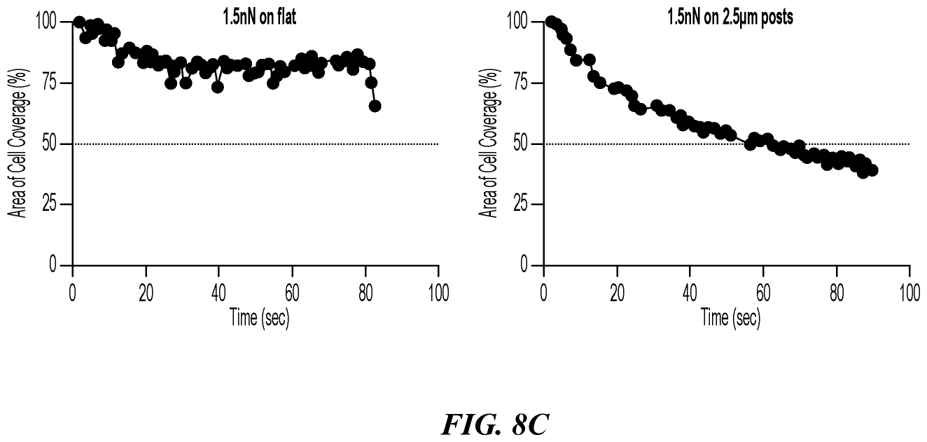

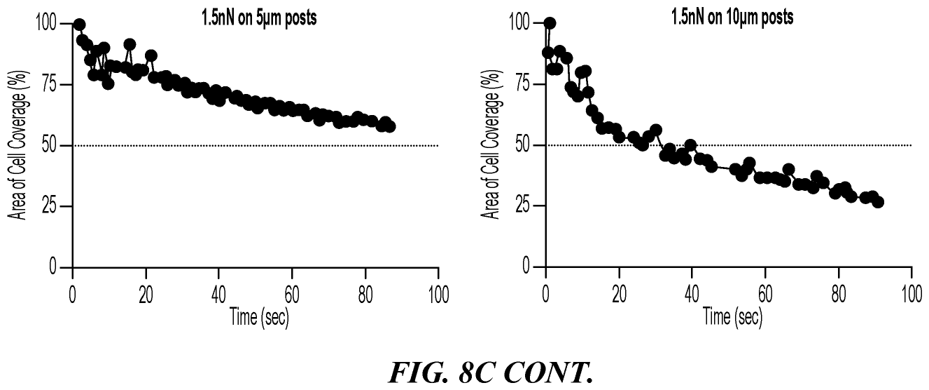

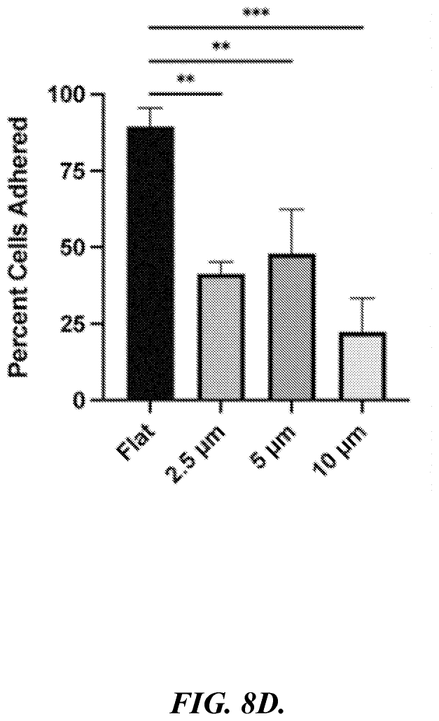

In order to confirm whether cells grown on the microtextured posts indeed have a lower cell adhesion strength, as observed using the contact ratio method, shear forces were applied to cells grown on the microtextured posts using a microfluidic system. MG-63 cells were seeded on microtextured PS surfaces within custom-built microfluidic chips (FIG. 15A) and allowed to grow for 24 h. The shear forces were then precisely controlled and applied to cells grown on the different micropost and flat control surfaces using a syringe pump to flow PBS buffer through the channel. A constant shear force of 1.5 nN was applied for 90 s. Videos were taken during the shear application (FIG. 8B, bright field images of t=0 s, and t=90 s with overlay), and the percent of cell confluency was determined at each time point using our lab created Python script (FIG. 8C,D). The flat surfaces showed the strongest adhesion, with an average of 10.5% of cells detached after 90 s of shear application. The post surfaces all showed statistically significant weaker adhesion, with the 2.5, 5, and 10 μm surfaces showing 58.6%, 45.1%, and 77.7% mean detachment, respectively, after 90 s (FIG. 8D). These results correspond with the observations in previous tests to validate that the cells grown on the micropost surfaces have lower adhesion strength compared to the flat control surface.

### Discussion

Human cancer cells grown on microtextured PS surfaces display no statistically significant deviations in their measured doubling time, proliferative capacity, cell turnover, and viability. The MG-63 and LNCaP cell lines maintained population doubling times of roughly 30 and 50 h, respectively. These values correspond with the reported doubling times for these cell lines of 28 and 48-60 h. Viability after passaging as measured by trypan blue exclusion remained at >95% for both cell lines on all surfaces. Similarly, proliferative capacity as measured by anti-Ki67 staining and EdU staining assay were expectedly high for MG-63 (85-95%+) and LNCaP (80-90%+). Results from both proliferative indices agreed with one another and values remained constant on all surfaces cells were cultured on. Cell turnover after one week in culture as measured by percent change in the area of fluorescently labeled dead cells showed a consistent ≈40% change in MG-63 cells and ≈20% change in LNCaP cells across all surfaces. The consistency in these results suggests that our microtextured surfaces are compatible for use with human cancer cell culture in a standard lab setting. These surfaces would likely be compatible with normal cell lines as well, however, cell types that are more sensitive to surface properties and environmental stimuli, such as stem cells, may respond differently to these microtextured surfaces. Thus, future studies would be required to investigate the impact of these microtextures on cell differentiation, in the case of stem cell culture.

Significant morphological changes were observed and quantified in MG-63 cells grown on microtextured PS surfaces. Cells cultured on our micropost surfaces, regardless of post dimensions, exhibited on average a roughly twofold reduction in population cell area and a twofold reduction in circularity as compared to a flat control surface. These cells also demonstrated an observably more elongated phenotype and measured almost twice as long to achieve the same cell area on a microtextured surface. Overall, the ratio of cell contact area to the post surface area varied slightly depending on the post dimensions, with the lowest ratio of ≈0.5 on 10 μm×10 μm, followed by ≈0.65 on 2.5 μm×2.5 μm, and finally ≈0.7 on 5 μm×5 μm. These differences were not statistically significant. However, as compared to cells cultured on a flat control surface (contact area ratio=1) MG-63 cells demonstrated a 30-50% reduction in surface contact area. Based on the findings regarding cell growth effects, the observed perturbations to cell shape are unlikely to have a negative impact on the population's overall growth in vitro. This may be in part due to the well-known mechanical plasticity and softness of cancer cells thus making them physically malleable, and therefore adaptable to such an environment.

Many techniques exist to attempt to probe the adhesion strength of single-cell attachments, as well as cell populations, however, each has limitations. Single-cell techniques, such as AFM, traction force microscopy, and micropipette manipulation assays, are typically low throughput and rely on complicated calculations to determine the adhesion forces. Cell population studies usually rely on the removal of cells by shear forces, which are likewise time-consuming, only working for weakly adherent cells, and are generally much less sensitive than single-cell measurements. Here, a quick, image recognition based technique to indirectly quantify the level of adhesion of cells to a microtextured surface was presented. This process allows for the comparison of the relative adhesion strengths of cells to each surface as a function of the surface morphology. The number of posts that a cell adheres to corresponds to the adhesion strength of the cell, with fewer adhesion points implying weaker cell attachment to the surface. Therefore, this information can be used to identify surface morphologies that enable both cell attachment and normal growth but with a smaller surface-contact area than on a flat surface.

In this study, a constant total effective surface area was maintained and significant differences due to variations in post dimensions were not observed. This suggests that the effective surface area plays a more important role in determining cell adhesion than post size and spacing. Thus, it would be expected that with a smaller effective surface area, such as in the case of larger post spacings, we would see lower cell-surface adhesion strength, and believe this is a direction that merits future research. A shear-based set-up to directly measure the relative adhesion of cells to each of the surfaces was also conducted. The shear data corresponds well with the image-based calculations, such that lower contact area results in lower adhesion strength. By applying equivalent shear forces to each surface for the same amount of time, a comparison can be made between the relative adhesion strength of cells to each surface. Interestingly, although the post densities differ between the micropost surfaces, the cells maintained the same level of adhesion strength. This could indicate that the accessible surface area or the spacing between microstructures are important geometric parameters that regulate cell-surface adhesion strength on microtextures, rather than the actual surface pattern.

However, further study is needed to definitively identify the properties governing cell-surface adhesion strength on microtextured surfaces. Surfaces that allow for the high growth and proliferation of cells, but low adhesion strength, can be valuable in many contexts. Typically, surfaces that are low-adhesion, such as hydrophobic surfaces or lubricant-impregnated surfaces (LIS), are designed to prevent cell adhesion. Such surfaces are useful for applications such as biofilm prevention or preventing buildup on implants. However, as surfaces such as these do not allow for cell growth and proliferation, they cannot be used for cell culture applications. The surfaces described with the entirety of this disclose could be used to culture cells while also making them more readily detachable using mechanical, non-enzymatic approaches. This is particularly helpful for primary cell cultures where trypsin-based detachment can be harmful to cell viability. These could also be used to grow skin grafts or other tissues for implantation by making these tissues easier to mechanically remove from the surfaces they are grown on and thus leading to less tissue damage during detachment. In addition, microtextures could also be used to mimic the human tissue microenvironment, enabling cell-matrix adhesion strengths to be tailored to match physiological conditions.

### Conclusion

A novel design process allowed for the the rapid fabrication of microtextured PS surfaces which demonstrated the biocompatibility of these surfaces. Using pure PS surfaces without any functionalized materials allowed us to observe and measure perturbations to cell morphology and adhesion as a direct consequence of surface morphology alone. In this study, the impact of PS micropost patterns with the same effective post-surface area on cell shape, adhesion, proliferation, death rates, doubling times, and viability were examined. While changes to cell morphology and adhesion were observed, these were not significantly impactful on the measured growth parameters we. Thus, these surfaces were shown to be amenable to long-term, continuous cell culture for use with established human cancer cell lines such as MG63 and LnCAP cells. However, due to the observed morphological changes and the reduced stiffness of cancer cells compared with normal cells, further work is needed to show if these observations will hold true for normal cell lines, primary cells, or stem cells.

These microtextured surfaces could be used to further modulate cell-surface adhesion as well. Future studies can investigate how changing the effective surface area of a microtextured surface impacts the adhesion strength of cells and examine whether the morphological changes to cell size and shape drive these changes in cell adhesion. These findings can also inform the design of low-adhesion surfaces to improve the speed and efficacy of existing cell detachment techniques, or even allow for the development of methods of rapid cell detachment without the need for trypsin or other enzymes.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A cell culture system configured to selectively affect cell adhesion and/or detachment, comprising:
a surface suitable for cell culture, the surface comprising a redox active species; and
an electric system configured to apply an alternating electrical field at or proximate the surface at an alternating frequency greater than or equal to 0.01 Hz and less than or equal to 0.1 Hz.

2. The cell culture system of claim 1, wherein the surface comprises a three-dimensionally patterned surface comprising a plurality of protrusions.

3. The cell culture system of claim 2, wherein the three-dimensionally patterned surface comprises a first material comprising the plurality of protrusions and a second material dispersed between the plurality of protrusions.

4. The cell culture system of claim 3, wherein the second material is configured to exhibit a morphological change upon exposure to the electric field.

5. The cell culture system of claim 4, wherein the first material comprises polystyrene.

6. The cell culture system of claim 1, wherein the surface comprises an electrically conductive polymer.

7. The cell culture system of claim 6, wherein the surface comprises polypyrrole, polyaniline, polythiophene, and/or poly(3,4-ethylenedioxythiophene) polystyrene sulfonate (PEDOT:PSS).

8. The cell culture system of claim 1, wherein the surface is optically transparent.

9. A cell culture system, comprising:
a vessel capable of being at least partially filled with a cell culture medium, the vessel comprising a first electrode in electrical communication with the cell culture medium when present; and
a surface, within the vessel and at least partially submerged with the cell culture medium when present, the surface being electrically coupled to the first electrode and configured to reduce the adhesion between the surface and cultured cells on the surface upon application of an AC voltage at a frequency capable of altering the morphology of cultured cells, wherein the surface comprises a redox active species.

10. The cell culture system of claim 9, wherein the surface comprises a three-dimensionally patterned surface comprising a plurality of protrusions.

11. The cell culture system of claim 10, wherein the spacing between each of the plurality of protrusions is greater than or equal to 2.5 micrometers and less than or equal to 10 micrometers.

12. The cell culture system of claim 10, wherein the plurality of protrusions comprise an upper geometric surface, the upper geometric surface comprising a surface area greater than or equal to 5 micrometers² and less than or equal to 100 micrometers².

13. The cell culture system of claim 10, wherein the three-dimensionally patterned surface comprises a first material comprising the plurality of protrusions and a second material interstitially dispersed between the plurality of protrusions.

14. The cell culture system of claim 13, wherein the second material is configured to exhibit a morphological change upon exposure to the electric field.

15. The cell culture system of claim 9, wherein the voltage comprises an AC voltage with a frequency greater than or equal to 0.01 Hz and less than or equal to 0.1 Hz.

16. The cell culture system of claim 9, wherein the voltage is greater than or equal to 0.1 V and less than or equal to 10 V.

17. A method for detaching cells from a surface, comprising:
applying an AC voltage at a frequency capable of altering the morphology of cultured cells across a first electrode and a surface, such that the adhesion between the surface and cultured cells on the surface is reduced,
wherein:
the first electrode is in electrical communication with the cell culture medium;
the surface comprises a redox active species; and
the surface is at least partially submerged with a cell culture medium when present.

18. The method for detaching cells from a surface of claim 17, further comprising agitating the cell culture medium, when present, such that cultured cells on the surface detach from the surface.

19. The method for detaching cells from a surface of claim 17, wherein the area of cell coverage on the surface after the application of the AC voltage for less than or equal to 30 seconds is less than or equal to 50% the area of cell coverage prior to the application of the AC voltage.

20. The method for detaching cells from a surface of claim 17, wherein the voltage comprises an AC voltage with a frequency greater than or equal to 0.01 Hz and less than or equal to 0.1 Hz.

21. (canceled)
